(19) United States
(12) Patent Application Publication
(10) Pub. No.: US 2008/0317793 A1
(43) Pub. Date: Dec. 25, 2008

(54) Method for Make-Up or Care of the Nails

(76) Inventor: Xavier Blin, Paris (FR)

Correspondence Address: OBLON, SPIVAK, MCCLELLAND MAIER & NEUSTADT, P.C., 1940 DUKE STREET, ALEXANDRIA, VA, 22314, US

(21) Appl. No.: 12/089,118

(22) PCT Filed: Oct. 2, 2006
(86) PCT No.: PCT/IB06/03863

§ 371 Date: May 9, 2008

Related U.S. Application Data
(60) Provisional application No. 60/722,415, filed on Oct. 3, 2005.

(51) Int. Cl. A61K 8/25; A61K 8/72; A61Q 3/00

(52) U.S. Cl. 424/401; 424/61

(57) ABSTRACT

The present invention relates to a method for make-up and/or care of the nails comprising gluing onto the nail, by means of an adhesive, a flexible sheet comprising at least one layer of at least one organic and/or inorganic material, the organic or inorganic material and/or the adhesive containing at least one silicone compound, said sheet having a thickness in the range from 1 μm to 2 mm. The invention also relates to a kit for make-up or care of the nails, as well as an article for make-up or care of the nails.

The present invention relates to a method of make-up and/or care of the nails or of false nails.

In general, make-up of nails or of false nails is restricting as it takes a long time. Thus, the user of nail varnish applies several layers of varnish, which have to be left to dry. After 3 to 5 days, the varnish flakes away and the gloss decreases—the user of nail varnish must remove the varnish and make-up her nails again.

Accordingly, in an endeavour to offer make-up compositions for nails or for false nails that are longer-lasting, nail varnish compositions have been proposed in the form of a kit of two liquid nail varnish compositions. However, the varnish is still not long-lasting, and the application of several layers is still restricting.

Furthermore, when nail varnish is coloured, its application is difficult and takes a long time, owing to the risk of spreading of the varnish onto the nail contour, and retouching on the nail using a solvent is difficult because it dissolves the areas that are correctly made-up as well as the areas requiring correction.

The present invention in fact aims to propose a method for make-up and/or care of the nails or of false nails which can overcome the drawbacks mentioned above, i.e. it lasts longer on the nails, and can be applied quickly and easily.

More precisely, the invention relates to a method for make-up and/or care of the nails comprising gluing onto the nail, using an adhesive, a flexible sheet comprising at least one layer of at least one organic and/or inorganic material, the organic and/or inorganic material and/or the adhesive containing at least one silicone compound, said sheet having a thickness in the range from 1 μm to 2 mm, and preferably in the range from 1 μm to 1.5 mm, and more preferably in the range from 10 μm to 1 mm.

Thickness, in the sense of the present invention, means the thickness before application on the nail of the whole of the inseparable structure of one or more layers comprising the organic and/or inorganic material.

Thus, if the flexible sheet is self-adhesive, the thickness is to mean the thickness of the sheet including the thickness of the adhesive.

Conversely, any structure fixed to the sheet in a detachable manner, in particular a protective film on either face of the sheet, in particular a silicone film on the adhesive face of the sheet, is not counted when measuring the thickness.

By organic and/or inorganic material, we mean an inorganic material, an organic material such as a polymer, or a hybrid or mixed organic/inorganic material.

The method according to the invention makes it possible to obtain a deposited film on the nails that is long-lasting, and in particular, owing to the presence of the silicone compound, has good resistance to abrasion and good resistance to water.

According to a particular embodiment, the invention relates to a method for make-up and/or care of the nails comprising gluing onto the nail, with the aid of an adhesive, a flexible sheet comprising at least one layer of at least one organic and/or inorganic material, the organic and/or inorganic material and the adhesive each containing at least one silicone compound.

According to one embodiment, the flexible sheet comprises a first face intended to be in contact with the nail, and a second face opposite the first, the adhesive being disposed on the first face of the sheet in such a way that the latter is adhesive. This embodiment will be designated by the term “adhesive sheet” hereinafter in this description.

It should be pointed out that in the case when the flexible sheet is adhesive, “thickness of the sheet” means the thickness of the sheet plus adhesive.

In the cases presented below in which the sheet and the adhesive are applied separately, “thickness of the sheet” means the thickness of the sheet alone.

According to another embodiment, the method comprises:
a. applying, on the nail, at least one layer of a liquid or solid composition containing said adhesive; and
b. applying, on the layer containing the adhesive, one face of said sheet so as to glue the latter to the nail.

According to another variant, the method according to the invention is characterized in that the sheet comprises a first face intended to be brought into contact with the nail and a second face opposite the first, said method comprising:
a. coating said first face of the sheet with a liquid or solid composition containing said adhesive; and
b. placing the first face thus coated in contact with the nail so as to glue said sheet on the nail.

In cases when the composition containing the adhesive is solid, it can for example be in the form of a film, both faces of which are coated with adhesive.

According to a second aspect, the invention also relates to an article for make-up and/or care of the nails comprising a flexible sheet with at least one layer of at least one organic and/or inorganic material, said sheet having a first adhesive face having an adhesive and intended to be brought into contact with the nail, and a second face opposite the first, the organic and/or inorganic material and/or the adhesive containing at least one silicone compound, said flexible sheet having a thickness in the range from 1 μm to 1 mm.

The invention also relates to an article for make-up and/or care of the nails comprising a flexible sheet with at least one layer of at least one organic and/or inorganic material, said sheet comprising a first adhesive face having an adhesive and intended to be brought into contact with the nail, and a second face opposite the first, the organic and/or inorganic material and the adhesive each containing at least one silicone compound.

In particular, the article can in addition have a protective film in contact with the first face of the flexible sheet, which is to be removed prior to placing the article on the nail. Preferably the face of the protective film in contact with the first face of the sheet is coated with a non-stick material, notably siliconized.

According to a particular embodiment, the article according to the invention is covered on both faces with a removable film, identical or different.

According to a third aspect, the invention relates to a kit for make-up and/or care of the nails comprising:
a. a flexible sheet with at least one layer of at least one organic and/or inorganic material, said flexible sheet having a first face intended to be brought into contact with the nail, and a second face opposite the first, said sheet having a thickness in the range from 1 μm to 2 mm,
b. an adhesive composition containing at least one adhesive, the adhesive and/or the organic and/or inorganic material containing at least one silicone compound.

According to one embodiment, the make-up kit according to the invention comprises the flexible sheet and the adhesive composition packaged separately.

The flexible sheet or the article, and in particular the surplus, can be precut or cut before or after its application, according to the desired size and shape, with small scissors, nail clippers or by scraping the film.

According to one embodiment, an additional layer of a liquid composition, such as a conventional nail varnish, comprising a film-forming polymer and an organic solvent, or “top coat”, is applied on the flexible sheet in order to improve the gloss of the latter.

By “flexible” we mean capable of deforming inelastically so as to match the more or less convex profile of the nail.

Advantageously, the flexible sheet is able to yield to mechanical deformations of the pulling type so as to adapt to the surface of a nail. This deformability is notably characterized by the parameter of breaking strain ∈_{b}, discussed later.

According to a preferred embodiment, the flexible sheet is removable. By “removable”, we mean that it can be removed when it is put in contact with conventional solvents, which are able to dissolve, at least partially, said organic and/or inorganic material and the adhesive. As examples, we may notably mention the polar organic solvents such as acetone, ester and/or short alcohol. In fact, in contact with a suitable solvent, the flexible sheet is able to swell, and this swelling aids its removal from the surface of a nail or of a false nail.

It also lasts a significant length of time and notably several days, preferably a week. The nail is thus provided with a film that is resistant to water, to rubbing and to impacts, and does not display significant wear or flaking during this period.

According to one embodiment, the flexible sheet according to the present invention can be in various shapes such as a star, a square, a circle etc.

According to yet another aspect, the invention relates to an article for make-up and/or care of the nails, as well as a method for make-up and/or care of the nails comprising applying, to the nails, an article comprising a flexible sheet with at least one layer of at least one organic and/or inorganic material, said sheet having a first adhesive face with an adhesive and intended to be brought into contact with the nail, and a second face opposite the first, the organic and/or inorganic material and/or the adhesive containing at least one silicone compound selected from
the pressure-sensitive silicone resins obtained by reaction of a silicone resin with reactive SiOH end groups and of a fluid polyorganosiloxane polymer of viscosity in the range from 1000 to 200000 cSt bearing reactive SiOH end groups,
the phenylsilicone resins with free silanol groups,
the so-called “room temperature vulcanization” reactive elastomeric silicones with adhesive and/or film-forming properties such as:
the silicone fluids or gums having alkoxysilane, acetoxysilane (or silanol) groups for in situ condensation/crosslinking in the presence of water and optionally a catalyst,
the silicone fluids or gums having SiH groups, designated “A”, mixed with silicones with CH═CH₂ unsaturated groups, designated “B”, with groups A reacting (crosslinking) with B on the substrate by hydrosilylation with a platinum or tin catalyst,
and mixtures thereof.

### Silicone Compound

The silicone compound can be selected from film-forming silicone compounds, silicone materials displaying adhesive properties, and mixtures thereof.

Among the silicone compounds mentioned below, some can display both film-forming and adhesive properties, for example according to their proportion of silicone or depending on whether they are used mixed with a particular additive. It is accordingly possible to vary the film-forming properties or the adhesive properties of such compounds according to the use envisaged, as in particular for the so-called “room temperature vulcanization” reactive elastomeric silicones described in sections I/ F/ and II/ A/ 4) below.

The silicone compound can be present in the flexible sheet and/or in the adhesive at a content in the range from 5 to 100 wt. %, preferably from 10 to 80 wt. %, more preferably from 15 to 50 wt. %, and even more preferably from 20 to 40 wt. % relative to the total weight of the layer containing it or of the adhesive.

### I/ Film-Forming Silicone Compounds

These film-forming compounds can be film-forming polymers or resins. By “film-forming” compound, we mean a compound that is able to form, on its own or in the presence of a film-forming auxiliary, a macroscopically continuous film on a substrate, for example keratinous materials or a polymer sheet (PET, nylon, polyolefin, cellulose etc.).

These film-forming compounds can be film-forming polymers or resins and can be selected from:

### A/ The Silicone Resins

These resins are polymers of crosslinked organosiloxanes.

The class of silicone resins is known by the name “MDTQ”, the resin being described according to the various siloxane monomer units that it contains, each of the letters “MDTQ” characterizing a type of unit.

The letter M represents the monofunctional unit of formula (CH₃)₃SiO_{1/2}, the silicon atom being joined to a single oxygen atom in the polymer containing this unit.

The letter D denotes a bifunctional unit (CH₃)₂SiO_{2/2} in which the silicon atom is joined to two oxygen atoms.

The letter T represents a trifunctional unit of formula (CH₃)SiO_{3/2}.

In the units M, D, T defined above, at least one of the methyl groups can be substituted with a group R different from the methyl group such as a hydrocarbon radical (notably alkyl) having from 2 to 10 carbon atoms or a phenyl group or a hydroxyl group.

Finally, the letter Q denotes a tetrafunctional unit SiO_{4/2} in which the silicon atom is joined to four hydrogen atoms which in turn are joined to the rest of the polymer.

Various resins with different properties can be obtained from these various units, the properties of these polymers varying depending on the type of monomers (or units), the type and number of substituted radicals, the length of the polymer chain, the degree of branching and the size of the pendent chains.

As examples of these silicone resins, we may mention:
a) The polyalkylsilsesquioxane resins which are resins essentially comprising units T and MT and preferably containing units “D”. We may mention for example the polymethylsilsesquioxane resins, the phenylpropylsilsesquioxane resins such as those marketed by the company Wacker under the reference Belsil SPR 45 VP which can provide shiny films, which can be obtained after dissolving in a volatile solvent such as butyl acetate or ethyl acetate,
b) The mixed resins comprising a combination of units M, T and Q, and which can optionally contain units D. Such resins are described for example in document US 2005/0186166, the contents of which are incorporated in the present application by reference.
   We may also mention the MQ-T propylsiloxane resins containing units:

(R¹₃SiO_{1/2})ₐ, (R²₂SiO_{2/2})_{b}, (R³SiO_{3/2})_{c}, and (SiO_{4/2})_{d}
in which R1, R2 and R3 represent independently a group selected from an alkyl group having from 1 to 8 carbon atoms, an aryl group, a carbinol group, an amino group, and
a is a number in the range from 0.05 to 0.5,
b is a number in the range from 0 to 0.3,
c is a number greater than 0
d is a number in the range from 0.05 to 0.6
with at least 40% of the groups R3 representing a propyl group,
as described in document WO 05/075542, the contents of which are incorporated in the present application by reference.
c) We may also mention the phenylsilicone resins containing free silanol groups, for example that marketed under the reference DC 217 Flake Resin. Such a polymer can provide shiny films, which can be obtained after dissolving in a volatile solvent such as butyl acetate or ethyl acetate.

### B/ Polymers Obtained by Supramolecular Assembly

By supramolecular polymer, we mean a polymer chain or network formed by the assembly of a polymer with at least one other polymer, each assembly containing at least one pair of matched joining groups. By pair of matched joining groups, we mean, in the sense of the invention, two joining groups each belonging to a polymer according to the invention and having at least three H bonds.

We may for example mention the polymers described in application WO 02/098377, the contents of which are incorporated in the present application by reference.

These supramolecular polymers can be silicone resins and/or polymers (crosslinked or not), vinyl/silicone or acrylic/silicone hybrid polymers, or silicone polycondensates.

### C/ Crosslinked Silicone Polymers and Organic/Silicone Hybrid Polymers (Crosslinked or Non-Crosslinked) Obtained by Sol Gel Technology.

Polymers obtained by this method are described for example in application WO 98/44906, the contents of which are incorporated in the present application by reference.

These polymers can be vinyl/silicone or acrylic/silicone hybrid polymers, or silicone polycondensates.

### D/ Organic/Silicone Hybrid Polymers

In addition to the aforementioned organic/silicone hybrid polymers specifically obtained by sol gel technology or by supramolecular assembly, we may mention:

1) Acrylic and vinylic grafted silicone copolymers or acrylic/silicone block copolymers comprising at least 3 blocks such as:
a) the fat-soluble acrylic grafted silicone polymers, for example the SA 70 polymers from 3M, alkylmethacrylate grafted polydimethylsiloxane copolymers such as KP 545 and 550 from the company Shin Etsu, or the grafted acrylic silicone polymers such as VS 70 and VS 80 from the company 3M,
b) the vinyl polymers having at least one unit derived from carbosiloxane dendrimer, preferably in the form of grafts; such polymers are described for example in documents EP0963751 or WO03/045337, the contents of which are incorporated in the present application by reference,
c) dispersions of particles of acrylic and vinyl polymers in an oil or an organic solvent and notably:
   particles of acrylic polymers, in dispersion in a liquid oil phase, and surface-stabilized with a stabilizer, selected from silicone polymers grafted with a hydrocarbon chain, such as described in document EP0749746,
   polymer particles of an ethylenic grafted silicone polymer, preferably an acrylic grafted silicone polymer, dispersed in a liquid oil phase, the ethylenic polymer being advantageously dispersed in the absence of additional surface stabilizer of the particles such as described notably in document EP 1428843,
d) aqueous dispersions of particles of acrylic/silicone hybrid polymers with co-crosslinking between the polyacrylate and the silicone such as those made by the company DAINIPPON INK & CHEMICAL and described in the document “Aqueous dispersion of polysiloxane/acrylic hybrid resins for coatings”, KUDO, in Chemical and polymer silicones in coatings—Conf Barcelona, Paper 31, pages 1-8,
and mixtures thereof.
   2) The polycondensates/silicones such as:
a) the silicone/polyurethane, silicone/polyurethane/urea, silicone/polyurea polyurethane, polyurethane/urea polycondensates and the polyurea polymers with silicone blocks for example
aqueous dispersions (or latices) of polyurethane/silicone multiblock polymers bearing ionic or ionizable groups, as described for example in documents EP 0751162 and EP 0782881
silicone/polyurethane block elastomers, preferably soluble in ethanol or in another polar solvent, for example those described in document WO 2003/014194 and marketed by the company Wacker under the reference Geniomer,
and mixtures thereof.
b) Silicone polyamides of the polyorganosiloxane type, for example those described in documents U.S. Pat. No. 5,874,069, U.S. Pat. No. 5,919,441, U.S. Pat. No. 6,051,216 and U.S. Pat. No. 5,981,680. We may mention for example the polyamide/polydimethysiloxane marketed under the reference DC 2-8179 by Dow Corning
   3) The polysaccharides, in particular the cellulose derivatives such as the cellulose esters, the cellulose ethers, preferably soluble in organic media and insoluble in water, nitrocellulose, said compounds having silicone grafts or sequences,
   and mixtures thereof.

### E/ The Dendrimers of Silicones Such as

a) silicone dendrimers or hyperbranched silicones with functional end groups (for example alkoxysilane or SiH groups) capable of co-crosslinking with polyacrylics bearing co-reactive functions,
b) hybrid particles of the core-shell type comprising an inorganic core and a silicone shell and optionally having chemically reactive functions capable of reacting with one another or with a different compound, for example with another silicone compound.
and mixtures thereof.
F/ The so-Called “Room Temperature Vulcanization” Reactive Elastomeric Silicones with Film-Forming Properties for Example
the silicone fluids or gums having alkoxysilane, acetoxysilane (or silanol) groups for condensation/crosslinking in situ in the presence of water and optionally a catalyst (for example a titanium derivative),
the silicone fluids or gums having SiH groups, designated “A”, mixed with silicones with unsaturated CH═CH₂ groups, designated “B”, the groups A reacting (crosslinking) with B on the substrate by hydrosilylation with a platinum or tin catalyst.

This platinum-catalysed reaction of hydrosilylation can take place:
either only by reaction of compound A with compound B (fluid or gum),
or by reaction of compound A with a mixture of B and a silicone resin (for example of the MQ or MT type) itself having reactive unsaturated —CH═CH₂ end groups. Presence or absence of the resin makes it possible to adjust the final film-forming properties of the system.
silicone latices with reactive groups for in situ crosslinking (alkoxysilanes or SiH groups/unsaturated group)
silicone adhesives without solvent, called “Hot Melt”. They are characterized by the solid state at temperatures below 180° F. and have low viscosities above 180° F. Consequently they can be formulated hot
silicone elastomers crosslinked with peroxides (very high temperature)

### II/ Adhesive Silicone Compounds

The adhesive used in the present invention can contain one or more adhesive silicone compounds, in particular one or more dissimilar silicone polymers. This adhesive can be in the form of a solution of polymer or of a dispersion of polymer particles in a solvent. The adhesive can also be in solid form, without solvent, and can be deposited hot on the flexible sheet by “hot melt” technology. This adhesive can in addition contain a plasticizer as defined below. This adhesive must exhibit a certain adhesive power defined by its viscoelastic properties.

The viscoelastic properties of a material are conventionally defined by the following two characteristics:

the elastic modulus which represents the elastic behaviour of the material at a given frequency and which is conventionally designated G′,
the viscosity modulus which represents the viscous behaviour of the material at a given frequency and which is conventionally designated G″.

These quantities are notably defined in the “Handbook of Pressure Sensitive Adhesive Technology” 3^{rd} edition, D. Satas, chap. 9, p. 155 to 157.

The adhesive materials that can be used according to the present invention display viscoelastic properties which are measured at a reference temperature of 35° C. and in a certain frequency range.

In the case of adhesive materials in the form of a solution or dispersion of polymer in a volatile solvent (such as water, a short ester, a short alcohol, acetone, a volatile silicone solvent, a volatile alkane etc.), the viscoelastic properties of the material are measured in conditions in which it has a content of volatile solvent below 30%, and in particular a content of volatile solvent below 20%.

In particular, the elastic modulus of the material is measured at three different frequencies:

at low frequency, i.e. at 2.10⁻² Hz,
at an intermediate frequency, i.e. at 0.2 Hz,
at high frequency, i.e. at 2 Hz,
and the viscosity modulus is measured at a frequency of 0.2 Hz.

These measurements make it possible to evaluate the variation of the adhesive power of the adhesive over time.

These viscoelastic properties are measured in dynamic tests under sinusoidal stresses of low amplitude (small strains) carried out at 35° C. over a frequency range from 2.10⁻² to 20 Hz on a type “Haake RS50®” rheometer under torsional/shear stress, for example in cone-plate geometry (for example with a cone angle of 1°).

Advantageously, said adhesive satisfies the following conditions:

G′(2 Hz, 35° C.)≧10³ Pa, and

G′(35° C.)≦10⁸ Pa, in particular G′(35° C.)≧10⁷ Pa,

G′(2.10⁻² Hz, 35° C.)≧3.10⁵ Pa,

in which:
G′(2 Hz, 35° C.) is the shear modulus of said adhesive, measured at a frequency of 2 Hz and temperature of 35° C.,
G′(35° C.) is the shear modulus of said adhesive, measured at a temperature of 35° C., for any frequency between 2.10⁻² and 2 Hz,
G′(2.10⁻² Hz, 35° C.) is the shear modulus of said adhesive, measured at a frequency of 2.10⁻² Hz and at a temperature of 35° C.

In a particular embodiment of the invention, the adhesive also satisfies the following condition:

−G″/G′(0.2 Hz, 35° C.)≧0.35.

in which:
G″ (0.2 Hz, 35° C.) is the viscosity modulus in shear of said adhesive, measured at a frequency of 0.2 Hz and a temperature of 35° C.,
G′(0.2 Hz, 35° C.) is the shear modulus of said adhesive, measured at a frequency of 0.2 Hz and a temperature of 35° C.

In a particular embodiment of the invention, we have:

−G′(2 Hz, 35° C.)≧5.10³ Pa, and in particular, G′(2 Hz, 35° C.)≧10⁴ Pa.

In another particular embodiment of the invention, we have:

−G′(2.10⁻² Hz, 35° C.)≦5.10⁴ Pa.

In particular, the adhesive materials according to the invention satisfy the following four conditions:

G′(2 Hz, 35° C.)≧10⁴ Pa, and

G′(35° C.)≦10⁸ Pa, in particular G′(35° C.)≦10⁷ Pa,

G′(2.10⁻² Hz, 35° C.)≦5.10⁴ Pa, and

G″/G′(0.2 Hz, 35° C.)≧0.35.

### A/ Silicone Resins and Elastomers Such as

1) Silicone resins containing combinations of units MQ and/or MT, (refer to section I/ for the list) and of units D as the main fraction, or resins containing combinations of units MQ and/or MT, mixed with silicone gums,
2) The “Pressure Sensitive Adhesives” for example:
compounds obtained by reaction of a silicone resin with free SiOH reactive end groups and a fluid polyorganosiloxane polymer of viscosity in the range from 1000 to 200000 cSt bearing SiOH reactive end groups, described for example in document U.S. Pat. No. 5,162,410 and marketed by the company Dow Corning under the name BIO PSA®, for example the compounds DC 7-4200, DC 7-43000, DC 7-4700, DC 7-4400, DC 7-4500 or DC 7-4600. These resins are therefore made up of units D, Q, and M,
mixtures of resins MQ and of polydimethylsiloxane gums, or mixtures of resins MQ and of silicone polyamides of the polyorganosiloxane type as described in section I/, D/2) b) above,
   3) Silicone elastomers such as the organopolysiloxanes crosslinked by hydrosilylation and notably the elastomeric organopolysiloxanes obtained by hydrosilylation of polydimethylsiloxanes with vinyl side and/or end groups, containing from 35 to 45 dimethylsiloxane units, by poly methylhydrogen-siloxane dimethylsiloxanes, containing at least two methylhydrogenosiloxane units and from 25 to 35, and even more preferably about 30 dimethylsiloxane units, for example those marketed under the designation KSG by the company Shin Etsu.

These silicone elastomers crosslinked by hydrosilylation can optionally bear hydrophilic groups such as hydrophilic polyoxyalkylene groups (in particular polyoxyethylene groups or polyoxyethylene/polyoxypropylene groups) and/or glycerol groups or polyglycerol groups,

4) The so-called “room temperature vulcanization” reactive elastomeric silicones with adhesive properties, for example
the silicone fluids or gums having alkoxysilane, acetoxysilane (or silanol) groups, for in situ condensation/crosslinking in the presence of water and optionally a catalyst (a titanium derivative, for example),
the silicone fluids or gums bearing SiH groups, designated “A”, mixed with silicones with CH═CH₂ unsaturated groups, designated “B”, with the groups A reacting (crosslinking) with B on the substrate by hydrosilylation with a platinum or tin catalyst.

This reaction of hydrosilylation with platinum catalysis can take place:
either only by reaction of compound A with compound B (fluid or gum)
or by reaction of compound A with a mixture of B and a silicone resin (for example of type MQ or MT) itself having —CH═CH₂ unsaturated reactive end groups. Presence or absence of the resin makes it possible to adjust the final adhesive properties of the adhesive system.
silicone latices with reactive groups for in situ crosslinking (alkoxysilanes or SiH groups/unsaturated group)
solvent-free silicone adhesives, called “Hot Melt”. They are characterized by a solid state at temperatures below 180° F. and display low viscosities above 180° F. Consequently they can be formulated hot
silicone elastomers crosslinked by peroxides (very high temperature)
   5) Polymers obtained by sol-gel reaction as described above, having a high proportion of silicone polymer.

### B/ The Organic/Silicone Hybrid Copolymers Such as:

1) The acrylic and vinylic silicone graft copolymers or the acrylic/silicone block copolymers comprising at least 3 blocks, said polymers having a high proportion of silicone.
2) The silicone polyurethanes and silicone polyamides with a high proportion of silicone.
3) It is also possible to select a polymer from the film-forming polymers mentioned in section I/ above, said polymer being modified to contain a high proportion of silicone so as to endow it with adhesive properties.

According to one embodiment, the organic and/or inorganic material of the flexible sheet contains at least one silicone compound preferably selected from the film-forming silicone polymers or resins mentioned above.

According to another embodiment, the adhesive contains at least one silicone adhesive material.

Advantageously, the organic and/or inorganic material of the flexible sheet and the adhesive each contain at least one silicone compound selected from those described above; preferably, the organic and/or inorganic material contains at least one silicone compound selected from the film-forming silicone polymers or resins mentioned above in section I/ and the adhesive contains at least one adhesive silicone compound such as those mentioned above in section II/.

According to an advantageous embodiment, the invention relates to a method for make-up and/or care of the nails comprising gluing to the nail, by means of an adhesive, a flexible sheet comprising at least one layer of at least one organic and/or inorganic material, the organic and/or inorganic material and/or the adhesive containing at least one silicone compound selected from:
the pressure-sensitive silicone resins obtained by reaction of a silicone resin with reactive SiOH end groups and a fluid polyorganosiloxane polymer of viscosity in the range from 1000 to 200000 cSt bearing reactive SIOH end groups,
the phenylsilicone resins with free silanol groups,
the so-called “room temperature vulcanization” reactive elastomeric silicones with adhesive and/or film-forming properties such as
   the silicone fluids or gums bearing alkoxysilane, acetoxysilane (or silanol) groups for condensation/crosslinking in situ in the presence of water and optionally a catalyst (for example a titanium derivative),
   the silicone fluids or gums bearing SiH groups, designated “A”, mixed with silicones with unsaturated CH═CH₂ groups, designated “B”, with groups A reacting (crosslinking) with B on the substrate by hydrosilylation with a platinum or tin catalyst.

### Flexible Sheet

Advantageously, the flexible sheet comprises at least one organic and/or inorganic material which is derived from polymerization/crosslinking of a polymerizable/crosslinkable composition containing at least said organic and/or inorganic material and/or evaporation of the organic or aqueous solvent phase, from a solution or dispersion of at least said material, preferably film-forming, which can be selected from the film-forming silicone compounds mentioned above and/or the non-silicone film-forming polymers which will be described later.

The flexible sheet (whether or not it is adhesive) employed in the present invention is a non-liquid film which can be characterized by a high dry extract. Thus, it can have a content of dry matter greater than 80%, and in particular greater than 85% and especially greater than 90 wt. % relative to its total weight. In other words the amount of volatile solvent is less than 20%, in particular less than 15% and more particularly less than 10 wt. % relative to the total weight of the flexible sheet.

Preferably, the content of dry matter, commonly called “dry extract”, of the films according to the invention, is measured by heating the test specimen with infrared radiation with wavelength from 2 μm to 3.5 μm. The substances contained in said films which possess a high vapour pressure evaporate under the action of this radiation. Measurement of the weight loss of the test specimen permits the “dry extract” of the film to be determined. These measurements are performed using a commercial LP16 infrared desiccator from Mettler. The method is fully described in the equipment documentation supplied by Mettler.

The measurement procedure is as follows.

About 10 g of the test specimen is placed in a metal dish. The latter is placed in the desiccator and is heated at a temperature of 120° C. for one hour. The wet mass of the test specimen, corresponding to the initial mass, and the dry mass of the test specimen, corresponding to the mass after exposure to the radiation, are measured using a precision balance.

The content of dry matter is calculated as follows:

Dry extract=100×(dry mass/wet mass).

According to a variant, the flexible sheet is not totally dry—it is said to be partially dry.

In the sense of the present invention, the term partially dry is intended to describe the fact that the flexible sheet obtained after evaporation of the organic or aqueous solvent phase, of a solution or dispersion of at least one film-forming polymeric material, or by polymerization/crosslinking of a polymerizable/crosslinkable composition, is not completely free of residual solvent. In particular, it has a content of dry matter less than 80%, in particular less than 75% and more particularly less than 70 wt. % relative to its total weight. According to a particular embodiment, the flexible sheet, in particular in the embodiment in which it is adhesive, is in a reservoir, for example a pocket, flexible or not, that is able to contain a product in hermetic conditions. It is in particular impermeable to air and/or to solvents. This packaging protects said sheet from drying completely and prematurely before it is used.

### Water Uptake

The flexible sheet employed in the invention can be characterized in the dry state by an uptake of water at 25° C. less than or equal to 20%, notably less than or equal to 16%, and in particular less than 10%.

According to the present application, by “water uptake” we mean the percentage of water absorbed by the film of the flexible sheet after 60 minutes of immersion in water, at 25° C. (room temperature). The water uptake is measured for pieces of about 1 cm² cut from the sheet, which are weighed (measurement of the mass M1) then immersed in water for 60 minutes; after immersion, the piece of sheet is wiped to remove excess surface water, then weighed (measurement of the mass M2). The difference M2−M1 corresponds to the amount of water absorbed by the film.

The water uptake is equal to [(M2−M1)/M1]×100 and is expressed in percentage by weight relative to the weight of the film.

### Storage Modulus E′

Moreover, the flexible sheet according to the invention advantageously has a storage modulus E′ greater than or equal to 1 MPa, notably in the range from 1 MPa to 5000 MPa, in particular greater than or equal to 5 MPa, notably in the range from 5 to 1000 MPa, and more particularly greater than or equal to 10 MPa for example in the range from 10 to 800 MPa at a temperature of 30° C. and a frequency of 0.1 Hz.

The storage modulus is measured by DMTA (Dynamical and Mechanical Temperature Analysis).

Viscoelasticimetry tests are carried out with the DMTA instrument from Polymer TA Instruments (model DMA2980), on a test specimen of sheet. The test specimens are cut out (for example with a punch). The test specimens typically have a thickness of about 150 μm, a width of 5 to 10 mm and a working length of about 10 to 15 mm.

The measurements are performed at a constant temperature of 30° C.

The test specimen is stressed in tension with small strains (for example it is submitted to a sinusoidal displacement of ±8 μm) during a frequency sweep, with frequency range from 0.1 to 20 Hz. We are thus operating in the linear domain, at low levels of strain.

These measurements allow us to determine the complex modulus E*=E′+iE″ of the film of test composition, E′ being the storage modulus and E″ the “loss” modulus.

Strain and/or Energy at Break

Advantageously, the flexible sheet according to the invention has, in the dry state, a breaking strain ∈_{b} greater than or equal to 5%, notably in the range from 5 to 500%, preferably greater than or equal to 15%, notably in the range from 15 to 400% and/or an energy at break per unit volume Wᵣ greater than or equal to 0.2 J/cm³, notably in the range from 0.2 to 100 J/cm³, preferably greater than 1 J/cm³, notably in the range from 1 to 50 J/cm³.

The breaking strain and the energy at break per unit volume are determined by tensile tests performed on a flexible sheet with thickness of about 200 μm.

For carrying out these tests, the sheet is cut into dumb-bell specimens with working length 33±1 mm and useful width of 6 mm. The cross-sectional area (S) of the test specimen is then defined as: S=width×thickness (cm²); this cross-sectional area will be used for calculating the stress.

The tests are carried out, for example, on a tensile tester marketed under the name Lloyd® LR5K. The measurements are performed at room temperature (20° C.).

The test specimens are stretched at a pulling speed of 33 mm/min, corresponding to a rate of 100% of elongation per minute.

Thus, a pulling speed is set, and the elongation ΔL of the test specimen and the force F required to produce this elongation are measured simultaneously. The parameters of stress σ and strain ∈ are determined from these data ΔL and F.

Thus, a curve of stress σ=(F/S) as a function of strain ∈=(ΔL/Lₒ)×100 is obtained, the test being continued until breakage of the test specimen, Lₒ being the initial length of the test specimen.

The breaking strain ∈_{b} is the maximum strain of the test specimen before the point of breakage (in %).

The energy at break per unit volume Wᵣ in J/cm³ is defined as the area under this stress/strain curve such that:

$${W}_{r} = {\int }_{0}^{{ɛ}_{r}} σ \cdot ɛ \cdot d ɛ$$

As explained above, the flexible sheet comprising at least one organic and/or inorganic material can be a film resulting from the polymerization/crosslinking of a polymerizable/crosslinkable composition containing at least said organic and/or inorganic material and/or a film resulting from the evaporation of the organic or aqueous solvent phase of a solution or dispersion of at least said organic and/or inorganic material, which is preferably film-forming, which can be selected from the film-forming silicone compounds mentioned above and/or the non-silicone film-forming polymers that will be described later.

### a) Polymerized/Crosslinked Film

In the sense of the present invention, a film described as polymerized/crosslinked can be fully or partially polymerized/crosslinked. In the case of partial crosslinking, the latter is of course sufficient to form the expected film. The reaction of polymerization/crosslinking will be designated hereinafter by the term “crosslinking”, for simplicity.

Of course, the compounds brought into contact with one another are selected, notably according to the nature of their respective functional groups, to be capable of interacting in the conditions of the crosslinking reaction in question.

This crosslinking can thus be carried out by thermal, photochemical and/or chemical means, in the presence or absence of a catalyst. Carrying out this crosslinking is within the competence of a person skilled in the art.
According to a first variant, the reaction of crosslinking is related to a reaction of polyaddition or polycondensation performed in the presence or absence of catalyst.

According to this first variant, the organic and/or inorganic film can notably result from the crosslinking of a reactive system formed by:

at least one first compound (A) having at least two functions (X), and
at least one second compound (B) having at least two functions (Y), reactive with respect to the functions X.

Advantageously, the reactive system possesses a mean functionality (total number of functions X and Y/total number of molecules of compounds (A) and (B)) greater than 2 so as to produce a three-dimensional network.

More particularly, to obtain a satisfactory crosslinking effect, the mean functionality of the reactive system can be at least equal to 2.2 and more particularly can vary from 2.5 to 100.

Compounds (A) and (B) can be of organic origin and can notably be of the monomer, oligomer, polymer and/or copolymer type or of an inorganic nature for example like a mineral particle, in which case they have the two required functions (X) or (Y) on the surface.

Functions X and Y, which are reactive with respect to one another, are selected from the so-called reactive functions and functions having at least one labile hydrogen.

More precisely, the reactive functions are selected from the isocyanate and epoxide functions and the ethylenic double bonds and the functions with labile hydrogen(s) are of the carboxylic, alcohol notably phenolic, primary or secondary amine, amide, aminoalcohol and/or thiol type.

According to this variant, compounds (A) and (B) brought into contact have respectively at least two so-called reactive functions of the epoxide and/or isocyanate type and at least two functions with labile hydrogen(s) notably of the amine or aminoalcohol type and can notably be selected from the compounds mentioned previously.

For example, X can be an epoxide and/or isocyanate function and Y can be selected from a carboxylic acid function and/or an anhydride function and/or an amine function and/or a thiol function and/or a hydroxyl function, in particular phenolic.

In this variant of the invention, crosslinking can be carried out by bringing together the compounds (A) and/or (B) having the functions (X) and/or (Y) in a blocked form and which can be deblocked beforehand or in the reaction conditions adopted for the crosslinking. This alternative is familiar to a person skilled in the art and will not be described in detail.

Compounds with Isocyanate Functions:

Compounds containing at least two free isocyanate functions are known in the prior art. They may be polyisocyanates, including diisocyanates or triisocyanates, which can have a molecular weight less than 500 000, or even less than 10 000. These polyisocyanates are generally obtained by polyaddition, polycondensation and/or grafting, bearing at least two isocyanate functions, either at the ends of the chain or on side groups.

The polyisocyanates can be linear, branched, aliphatic, cycloaliphatic or aromatic.

The polyisocyanate used can in particular be DESMODUR® N from the company BAYER, or TOLONATE® HDB-LV from the company RHODIA.

Compounds with Epoxide Functions:

Compounds having at least two epoxide functions are also known from the prior art. They can be of any chemical nature. They can be diepoxides or polyepoxides of low mass (less than or equal to 5000), or oligomers or polymers of any chemical nature, obtained by polyaddition, polycondensation and/or grafting, bearing at least two free epoxide functions, either at the ends of chains, or in side groups.

Polymers with epoxy functions are marketed under the designations CYRACURE® UVR-6110, CYRACURE® UVR-6105, CYRACURE® ERL-4221E, CYRACURE® ERL-4206, CYRACURE® UVR 6128, CYRACURE® UVR 6216 by the company UNION CARBIDE, DER® 439 by the company DOW CHEMICAL, the EPIKATES® 828, 1001, 1004, 1007 from the company SHELL, ARALDITE® ECN1299 from the company CIBA-GEIGY, and EPOXYNOVOLACS® from the company DOW CHEMICAL.

Compounds with Ethylenic Double Bonds:

Compounds with ethylenic double bonds can be of any chemical nature. They can notably be selected from:

Polyesters with Side and/or Terminal (Meth)Acrylate Groups:

Such polyesters are marketed for example by the company UCB under the designations EBECRYL® (EBECRYL® 450: molar mass 1600, on average 6 acrylate functions per molecule, EBECRYL® 652: molar mass 1500, on average 6 acrylate functions per molecule, EBECRYL® 800: molar mass 780, on average 4 acrylate functions per molecule, EBECRYL® 810: molar mass 1000, on average 4 acrylate functions per molecule, EBECRYL® 50 000: molar mass 1500, on average 6 acrylate functions per molecule).

Polyurethanes and/or Polyureas with (Meth)Acrylate Groups Notably Obtained by Polycondensation:

Such polyurethanes/polyureas with acrylate groups are marketed for example under the designation SR 368 (tris(2-hydroxyethyl)isocyanurate-triacrylate) or CRAYNOR® 435 by the company CRAY VALLEY, or under the designation EBECRYL® by the company UCB (EBECRYL® 210: molar mass 1500, 2 acrylate functions per molecule, EBECRYL® 230: molar mass 5000, 2 acrylate functions per molecule, EBECRYL® 270: molar mass 1500, 2 acrylate functions per molecule, EBECRYL® 8402: molar mass 1000, 2 acrylate functions per molecule, EBECRYL® 8804: molar mass 1300, 2 acrylate functions per molecule, EBECRYL® 220: molar mass 1000, 6 acrylate functions per molecule, EBECRYL® 2220: molar mass 1200, 6 acrylate functions per molecule, EBECRYL® 1290: molar mass 1000, 6 acrylate functions per molecule, EBECRYL® 800: molar mass 800, 6 acrylate functions per molecule).

We may also mention the water-soluble aliphatic diacrylate polyurethanes marketed under the designations EBECRYL® 2000, EBECRYL® 2001 and EBECRYL® 2002, and the diacrylate polyurethanes in aqueous dispersion marketed under the designations IRR® 390, IRR® 400, IRR® 422 IRR® 424 by the company UCB.

polyethers with (meth)acrylate groups obtained by esterification, with (meth)acrylic acid, of the terminal hydroxyl groups of homopolymers or of copolymers of C₁₋₄ alkyleneglycols, such as polyethyleneglycol, polypropyleneglycol, the copolymers of ethylene oxide and propylene oxide preferably having a weight-average molecular weight less than 10 000, and polyethoxylated or polypropoxylated trimethylolpropane.

Di(meth)acrylate polyoxyethylenes of suitable molar mass are marketed for example under the designations SR 259, SR 344, SR 610, SR 210, SR 603 and SR 252 by the company CRAY VALLEY or under the designation EBECRYL® 11 by UCB. Triacrylates of polyethoxylated trimethylolpropane are marketed for example under the designations SR 454, SR 498, SR 502, SR 9035, SR 415 by the company CRAY VALLEY or under the designation EBECRYL® 160 by the company UCB. Triacrylates of polypropoxylated trimethylolpropane are marketed for example under the designations SR 492 and SR 501 by the company CRAY VALLEY.

Epoxyacrylates such as those marketed for example under the designations SR 349, SR 601, CD 541, SR 602, SR 9036, SR 348, CD 540, SR 480, CD 9038 by the company CRAY VALLEY, under the designations EBECRYL® 600 and EBECRYL® 609, EBECRYL® 150, EBECRYL® 860, EBECRYL® 3702 by the company UCB and under the designations PHOTOMER® 3005 and PHOTOMER® 3082 by the company HENKEL.

poly(meth)acrylates of (C₁₋₅₀ alkyl) having at least two functions with ethylenic double bonds carried by side or terminal hydrocarbon chains.

Such copolymers are marketed for example under the designations IRR® 375, OTA® 480 and EBECRYL® 2047 by the company UCB.

Dendrimers and hyperbranched polymers bearing (meth)acrylate or (meth)acrylamide end groups notably obtained respectively by esterification or amidification of dendrimers and of hyperbranched polymers with hydroxyl or amino terminal functions, by (meth)acrylic acid. Dendrimers (from the Greek dendron=tree) are “arborescent” polymeric molecules, i.e. highly branched, invented by D. A. Tomalia and his team at the beginning of the 1990s (Donald A. Tomalia et al., Angewandte Chemie, Int. Engl. Ed., vol. 29, No. 2, pages 138-175). They are structures built around a generally polyvalent central unit. Branched chain-extension units are arranged around this central unit, according to a fully defined structure, thus giving rise to symmetrical, monodispersed macromolecules, having a well-defined chemical and stereochemical structure. Dendrimers of the polyamidoamine type are marketed for example under the designation STARBUST® by the company DENDRITECH. The hyperbranched polymers are polycondensates, generally of the polyester, polyamide or polyethyleneamine type, obtained from polyfunctional monomers, which have an arborescent structure similar to that of the dendrimers but much more regular than the latter (see for example WO-A-93/17060 and WO 96/12754).

The company PERSTORP markets hyperbranched polyesters under the designation BOLTORN®. Hyperbranched polyethyleneamines are available under the designation COMBURST® from the company DENDRITECH. Hyperbranched poly(esteramide)s with hydroxyl end groups are marketed by the company DSM under the designation HYBRANE®. These dendrimers and hyperbranched polymers esterified or amidified with acrylic and/or methacrylic acid differ from the polymers described in the above paragraphs a) to h) by the very large number of ethylenic double bonds present.

This high functionality, generally greater than 5, makes them particularly useful so that they can act as a “crosslinking node”, i.e. a multiple crosslinking site.

In a preferred embodiment of the invention, these dendritic and hyperbranched polymers will consequently be used in conjunction with one or more of the above polymers and/or oligomers a) to h).

Compounds Bearing at Least Two Functions with Labile Hydrogen(s)

Compounds bearing at least two functions with labile hydrogens that can be used in the present invention are also known. They may be low molecular weight organic compounds or oligomers or synthetic polymers, obtained by polyaddition, polycondensation and/or grafting, or chemically modified natural polymers.

According to the present invention, functions with a labile hydrogen are preferably selected from the following functions: primary amine (—NH₂), secondary amine (>NH), hydroxyl (—OH), carboxylic acid (—COOH) or thiol (—SH).

When the function with a labile hydrogen is a hydroxyl function, we may mention as classes of compounds, the aliphatic diols and polyols.

When the function with a labile hydrogen is an amine function (NH₂), it can be a diamine, a polyamine, an aminoalcohol, an oligomer, or a polymer with amine groups.

Particular examples of compounds bearing functions with labile hydrogens are: the C₁₋₄ alkyleneglycols, glycerol, trimethylolpropane, pentaerythritol, poly(C₁₋₄ alkylene)glycols such as polyethyleneglycol or polypropyleneglycol or copolymers of the latter, the product of condensation of propyleneglycol and trimethylolpropane, castor oil, phytantriol, sugars and carbohydrates such as sucrose or cellulose, ethylenediamine, 1,3-diaminopropane, lysine, amino-2-methyl-2-propanol-1, poly(alkyleneoxy)diamines such as the JEFFAMINE® products marketed by the company TEXACO, nitrocellulose, cellulose esters, notably those having a degree of substitution less than 3, such as cellulose acetobutyrate and cellulose acetopropionate, cellulose ethers such as hydroxyethylcellulose, carboxymethylcellulose, hydroxypropylcellulose or ethylcellulose, polyester resins, perfluoropolyethers, alkyds and polyketones with hydroxylated ends, poly(vinyl alcohol) and copolymers based on vinyl alcohol, copolymers of allyl alcohol, copolymers based on C₂₋₁₀ hydroxyalkyl (meth)acrylate, such as 2-hydroxyethyl or 2-hydroxypropyl (meth)acrylate, notably sold under the designation JONCRYL® SCX 910 by the company JOHNSON POLYMER or under the designation CRODOPLAST® AC 5725 by the company CRODA, copolymers based on vinylamine or allylamine, perfluoroethers with primary or secondary amine ends, dendrimers or hyperbranched polymers with hydroxyl or primary amine ends such as hyperbranched polyesters with hydroxyl ends marketed by the company PERSTORP under the designations BOLTORN® H40 TMP CORE and HBP POLYOL® 3G (described in international applications WO 93/17060 and WO 96/12754), or dendrimers of the polyamido-amine types with primary amine ends described in the article of Tomalia, Angewandte Chemie, Int. Engl. Ed., vol. 29, No. 2, pages 138-175.

According to a second variant of the invention, crosslinking is carried out photochemically and employs at least two types of compounds notably (A) and (B) having respectively at least one unsaturated double bond in the presence of a photoinitiator.

According to this variant, A and B are selected such as to form a reactive system whose average valence is greater than 2. The valence of a compound means the number of covalent bonds that it can establish with the compounds that are associated with it. The average valence is defined as being equal to the ratio of the sum of the valences of all the compounds A and B divided by the total number of compounds A and B

$${V}_{m} = \frac{\sum nivi}{\sum ni}$$

According to this variant of the invention, compounds A or B can be a compound comprising a function of the unsaturated double bond type and notably as defined above, and/or a monomer with ethylenic unsaturation.

A particular group of advantageous photoinitiators according to the invention is that of the copolymerizable photoinitiators. They are molecules comprising both a photoinitiator group capable of a photo-induced radical cleavage and at least one ethylenic double bond.

To obtain satisfactory lasting properties, generally the total amount of photoinitiator(s) used is at least equal to 0.1 wt. % and at most equal to 10 wt. %, and preferably between 0.2 and 5 wt. %, relative to the total weight of compounds comprising ethylenic double bonds.

In this variant, crosslinking can be carried out in the presence of a co-film-forming agent, for example nitrocellulose or cellulose esters.

b) Film Resulting from the Evaporation of the Organic or Aqueous Solvent Phase of a Solution or Dispersion of at Least One Organic and/or Inorganic Material.

According to a second variant of the invention, the flexible sheet is an organic and/or inorganic film obtained by evaporation of the organic or aqueous solvent phase, of a solution or dispersion of at least said, preferably film-forming material, selected from the film-forming silicone compounds mentioned above and/or the non-silicone film-forming polymers hereunder.

The non-silicone film-forming polymer can be selected from the group comprising radical polymers, polycondensates and polymers of natural origin.

In the case when the sheet is not adhesive, the film can be obtained by application of the second composition on a substrate coated with Teflon®, then drying at a temperature in the range from 20 to 150° C. The film is then detached from the substrate and is then glued using a solid or liquid adhesive.

### Non-Silicone Film-Forming Polymers Soluble or Dispersible in an Organic Solvent

According to a first variant, the organic and/or inorganic film of the sheet is derived from the evaporation of the organic solvent phase of a solution or dispersion of at least one film-forming material. In this embodiment, the film-forming organic polymer is at least one polymer selected from the group comprising: the film-forming polymers soluble or dispersible in at least one class of organic solvent such as for example the ketones, the alcohols, the glycols and ethers of propylene glycols, the short-chain esters, the alkanes and mixtures thereof, aqueous or non-aqueous.

The corresponding polymers can be of any chemical nature. In particular, they can result either from the homo- or co-polymerization of unsaturated monomers, or from polycondensation, or from the modification of natural polymers, in particular polysaccharides. The weight-average molecular weights (Mw) of these polymers can range from 3000 to 1 000 000, notably from 5000 to 800 000, and in particular from 10 000 to 500 000.

Among the polymers soluble or dispersible in organic solvents, the following polymers are quite particularly suitable:

a) The homo- and co-polymers that are esters and/or amides of (meth)acrylic acids, in particular the polymers resulting from the polymerization or copolymerization of the methyl, ethyl, propyl, butyl, isobutyl, tert-butyl, pentyl, hexyl, cyclohexyl, ethyl 2-hexyl, heptyl, octyl, isobornyl, norbornyl, adamantyl acrylates and/or methacrylates, or the corresponding (meth)acrylamides. These polymers will preferably have from 0 to 20% of a polar comonomer such as (meth)acrylic acid, (meth)acrylamide, hydroxyethyl (meth)acrylate, 2-hydroxypropyl (meth)acrylate, and (meth)acrylonitrile. They can also result from copolymerization with styrene or a substituted styrene.
b) The homo- and co-polymers that are vinylic esters or amides, in particular the homo- and co-polymers resulting from the polymerization of vinyl acetate, vinyl propionate, vinyl versatate, with or without the presence of a polar comonomer such as crotonic acid, allyloxyacetic acid, maleic anhydride (or acid), itaconic anhydride (or acid), vinyl acetamide and vinyl formamide. Moreover, they can result from the copolymerization of at least one of the aforementioned monomers with styrene or a substituted styrene.
c) Celluloses and cellulose derivatives such as nitrocelluloses and/or cellulose esters such as cellulose acetates, cellulose propionates, cellulose butyrates, cellulose acetopropionates and cellulose acetobutyrates.
d) The polycondensates soluble or dispersible in these solvents. They are generally used as principal film-forming agent or as co-film-forming agent of one of the classes of polymers mentioned previously (a to c), especially if they are of low molecular weight (Mw<20 000). They can be selected from the following polymers or copolymers: polyurethanes, acrylic polyurethanes, polyureas, polyurea polyurethanes, polyester polyurethanes, polyether polyurethanes, polyesters, polyester-amides, aliphatic-chain polyesters, epoxides, and arylsulphonamide condensates and in particular tosylamide/formaldehydes.

Among these polycondensates, in particular if one or more nitrocelluloses and/or a cellulose ester (class c) are used as film-forming or co-film-forming agent, we may more particularly mention:

the polyesters, in particular the aliphatic-chain polyesters and more particularly the copolymers with the CTFA name: “copolymer of phthalic anhydride/glycerol/glycidyl decanoate” and “copolymer of adipic acid/neopentylglycol/trimellitic anhydride”
the alkyds,
the tosylamide/formaldehyde condensates,
the polyurethanes and polyurea-urethanes,
the acrylic resins.

According to one embodiment of the invention, the film-forming polymer is a linear sequenced ethylenic film-forming polymer, comprising preferably at least one first sequence and at least one second sequence having different glass transition temperatures (Tg), said first and second sequences being joined together by an intermediate sequence comprising at least one monomer constituting the first sequence and at least one monomer constituting the second sequence.

Advantageously, the first and second sequences and the sequenced polymer are incompatible with one another.

Such polymers are described for example in documents EP1411069 or WO04/028488.

### Aqueous Dispersions of Particles of Polymers or of Film-Forming Latices

According to a second variant of the invention, said organic and/or inorganic film results from the evaporation of the aqueous phase of an aqueous dispersion of particles of film-forming polymer(s). In this case, the film-forming polymer can be selected from the aqueous dispersions of particles of polymers or of film-forming latices, and in this case the composition according to the invention comprises at least one aqueous phase.

The aqueous dispersion comprising one or more film-forming polymers can be prepared by a person skilled in the art on the basis of his general knowledge, notably by emulsion polymerization or by dispersion of the previously formed polymer.

Among the film-forming polymers of this type that can be used in the composition according to the present invention, we may mention synthetic polymers, of the polycondensate or radical type, polymers of natural origin, and mixtures thereof.

Notably it is possible to use, but in the form of latex, the polymers (homo- and co-polymers) which have already been mentioned as polymers soluble or dispersible in organic solvent, and more particularly the polymers of classes a, b and c.

Thus, we may mention, among the polycondensates, the anionic, cationic, non-ionic or amphoteric polyurethanes, polyurethane-acrylics, polyurethane-polyvinylpyrrolidones, polyester-polyurethanes, polyether-polyurethanes, polyureas, polyurea polyurethanes, and mixtures thereof.

We may also mention polyesters, polyester-amides, aliphatic-chain polyesters, polyamides and epoxyester resins.

The polyesters can be obtained, in a known manner, by polycondensation of aliphatic or aromatic diacids with aliphatic or aromatic diols or polyols. Succinic acid, glutaric acid, adipic acid, pimelic acid, suberic acid or sebacic acid can be used as aliphatic diacids. Terephthalic acid or isophthalic acid, or a derivative such as phthalic anhydride, can be used as aromatic diacids. Ethylene glycol, propylene glycol, diethylene glycol, neopentyl glycol, cyclohexane dimethanol, 4,4′-(1-methylpropylidene)bisphenol can be used as aliphatic diols. Glycerol, pentaerythritol, sorbitol, and trimethylol propane can be used as polyols.

The polymers of the radical type can notably be acrylic and/or vinylic polymers or copolymers. Anionic radical polymers are preferably used. As monomer bearing an anionic group that can be used in radical polymerization, we may mention acrylic acid, methacrylic acid, crotonic acid, maleic anhydride, acrylamido-2 methyl-2 propane sulphonic acid.

The acrylic polymers can result from the copolymerization of monomers selected from the esters and/or the amides of acrylic acid or of methacrylic acid. As examples of monomers of the ester type, we may mention methyl methacrylate, ethyl methacrylate, butyl methacrylate, isobutyl methacrylate, ethyl-2 hexyl methacrylate, lauryl methacrylate. As examples of monomers of the amide type, we may mention N-t-butyl acrylamide and N-t-octyl acrylamide. The vinyl polymers can result from the homopolymerization or the copolymerization of monomers selected from the vinyl esters, styrene or butadiene. As examples of vinyl esters, we may mention vinyl acetate, vinyl neodecanoate, vinyl pivalate, vinyl benzoate and vinyl t-butyl benzoate.

We may also mention the polymers resulting from the radical polymerization of one or more radical monomers within and/or partially on the surface, of pre-existing particles of at least one polymer selected from the group comprising the polyurethanes, the polyureas, the polyesters, the polyester amides and/or the alkyds. These polymers are generally called hybrid polymers.

The dispersion can also comprise an associative polymer of the polyurethane type or a natural gum, such as xanthan gum.

As polymer in aqueous dispersion, we may mention the dispersions of acrylic polymers sold under the designations NEOCRYL XK-90®, NEOCRYL A-1070®, NEOCRYL A-1090®, NEOCRYL BT-62®, NEOCRYL A-1079®, NEOCRYL A-523® by the company ZENECA, DOW LATEX 432®, by the company DOW CHEMICAL. We may also use aqueous dispersions of polyurethane, and notably the polyester-polyurethanes sold under the designations “AVALURE UR-405®”, “AVALURE UR-410®”, “AVALURE UR-425®”, “SANCURE 2060®” by the company GOODRICH and the polyether-polyurethanes sold under the designations “SANCURE 878®” by the company GOODRICH, “NEOREZ R-970®” by the company AVECIA.

All of the aforementioned film-forming polymers can be combined with at least one film-forming auxiliary.

The film-forming auxiliary can be selected from all the compounds known by a person skilled in the art as being capable of fulfilling the required function, and can notably be selected from plasticizers and coalescence agents of the film-forming polymer.

In particular, we may mention, alone or as mixtures, the usual plasticizers or coalescence agents, such as:

the glycols and their derivatives such as diethylene glycol ethylether, diethylene glycol methylether, diethylene glycol butylether or diethylene glycol hexylether, ethylene glycol ethylether, ethylene glycol butylether, ethylene glycol hexylether;
the glycol esters,
the derivatives of propylene glycol and in particular propylene glycol phenylether, propylene glycol diacetate, dipropylene glycol butylether, tripropylene glycol butylether, propylene glycol methylether, dipropylene glycol ethylether, tripropylene glycol methylether and diethylene glycol methylether, propylene glycol butylether,
the esters of acids, notably carboxylic, such as the citrates, notably triethyl citrate, tributyl citrate, triethyl acetylcitrate, tributyl acetylcitrate, triethyl-2 hexyl acetylcitrate; phthalates, notably diethyl phthalate, dibutyl phthalate, dioctyl phthalate, dipentyl phthalate, dimethoxyethyl phthalate; phosphates, notably tricresyl phosphate, tributyl phosphate, triphenyl phosphate, tributoxyethyl phosphate; tartrates, notably dibutyl tartrate; adipates; carbonates; sebacates; benzyl benzoate, butyl acetylricinoleate, glyceryl acetylricinoleate, butyl glycolate, camphor, glycerol triacetate, N-ethyl-o,p-toluenesulphonamide,
the oxyethylenated derivatives such as oxyethylenated oils, notably vegetable oils such as castor oil; silicone oils,
mixtures thereof.

The type and the amount of plasticizer and/or coalescence agent can be selected by a person skilled in the art on the basis of his general knowledge.

For example, the content of plasticizer and/or coalescence agent can range from 0.01 to 20 wt. % and in particular from 0.5 to 10 wt. % relative to the total weight of the composition.

### c) Organic or Aqueous Solvent Phase

When the flexible sheet comprising at least one organic and/or inorganic material results from evaporation of the organic or aqueous solvent phase of a solution or dispersion of at least said, preferably film-forming, material, the solvent phase can be:
a liquid organic solvent phase comprising at least one organic solvent selected from:
   short-chain esters (having from 3 to 8 carbon atoms in total), such as ethyl acetate, methyl acetate, propyl acetate, n-butyl acetate, isopentyl acetate;
   ketones that are liquid at room temperature, such as methylethylketone, methylisobutylketone, diisobutylketone, isophorone, cyclohexanone, acetone;
   alcohols that are liquid at room temperature, such as ethanol, isopropanol, diacetone alcohol, 2-butoxyethanol, cyclohexanol;
   glycols that are liquid at room temperature, such as ethylene glycol, propylene glycol, pentylene glycol, glycerol;
   propylene glycol ethers that are liquid at room temperature, such as propylene glycol monomethylether, propylene glycol monomethyl ether acetate, dipropylene glycol mono n-butyl ether;
   aldehydes that are liquid at room temperature, such as benzaldehyde, acetaldehyde;
   carbonates such as propylene carbonate, dimethyl carbonate;
   acetals such as methylal; and

A volatile hydrocarbon and/or silicone solvent can also be used advantageously.

By “volatile solvent”, we mean in the sense of the invention a solvent (or oil) that can evaporate in contact with the skin or keratin fibre in less than one hour, at room temperature and atmospheric pressure. The volatile organic solvent or solvents are liquid at room temperature and have a non-zero vapour pressure, at room temperature and atmospheric pressure, in particular in the range from 0.13 Pa to 40 000 Pa (10⁻³ to 300 mmHg), in particular in the range from 1.3 Pa to 13 000 Pa (0.01 to 100 mmHg), and more particularly in the range from 1.3 Pa to 1300 Pa (0.01 to 10 mmHg).

Conversely, “non-volatile solvent” means a solvent that remains on the nails at room temperature and atmospheric pressure for at least several hours and notably has a vapour pressure below 10⁻³ mmHg (0.13 Pa).

The volatile hydrocarbon solvents can be selected from the hydrocarbon solvents having from 8 to 16 carbon atoms, and notably the C₈-C₁₆ branched alkanes such as the C₈-C₁₆ isoalkanes of petroleum origin (also called isoparaffins) such as isododecane (also called 2,2,4,4,6-pentamethylheptane), isodecane, isohexadecane, and for example the solvents sold under the trade names Isopars' or Permethyls, the C₈-C₁₆ branched esters such as isohexyl neopentanoate, and mixtures thereof. Other volatile hydrocarbon solvents such as the petroleum distillates, notably those sold under the designation Shell Solt by the company SHELL, can also be used. Preferably, the volatile solvent is selected from the volatile hydrocarbon solvents having from 8 to 16 carbon atoms and mixtures thereof.

The volatile silicones used can be for example volatile linear or cyclic silicone solvents, notably those having a viscosity ≦8 centistokes (8 10⁻⁶ m²/s), and notably having from 2 to 7 silicon atoms, these silicones optionally bearing alkyl or alkoxy groups having from 1 to 10 carbon atoms. As volatile silicone solvent for use in the invention, we may notably mention octamethyl cyclotetrasiloxane, decamethyl cyclopentasiloxane, dodecamethyl cyclohexasiloxane, heptamethyl hexyltrisiloxane, heptamethyloctyl trisiloxane, hexamethyl disiloxane, octamethyl trisiloxane, decamethyl tetrasiloxane, dodecamethyl pentasiloxane and mixtures thereof.

We may also mention the volatile linear alkyltrisiloxane oils of general formula (I):

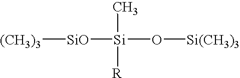

Where R represents an alkyl group having from 2 to 4 carbon atoms and in which one or more hydrogen atoms can be substituted with a fluorine or chlorine atom.

Among the oils of general formula (I), we may mention:
3-butyl 1,1,1,3,5,5,5-heptamethyl trisiloxane,
3-propyl 1,1,1,3,5,5,5-heptamethyl trisiloxane, and
3-ethyl 1,1,1,3,5,5,5-heptamethyl trisiloxane,

corresponding to the oils of formula (I) for which R is respectively a butyl group, a propyl group or an ethyl group.

Volatile fluorinated solvents such as nonafluoromethoxybutane or perfluoromethylcyclopentane can also be used.

Preferably, the solvent is a volatile solvent selected from the short-chain esters (having from 3 to 8 carbon atoms in total) such as ethyl acetate, methyl acetate, propyl acetate, n-butyl acetate, isopentyl acetate, alkanes having from 8 to 16 carbon atoms, volatile silicones and mixtures thereof.

Generally, the liquid organic solvent phase (organic solvent or mixture of organic solvents) represents from 5 to 95% of the total weight of the initial composition (before evaporation), preferably from 10 to 85 wt. %.
an aqueous phase constituted of water and optionally of water-soluble solvents, and in this case the aqueous phase can represent from 5 to 95 wt. % relative to the total weight of the composition, preferably from 10 to 85%, preferably from 15 to 60 wt. %.

According to a particular embodiment, the flexible sheet is a multilayer film made in several stages from different organic and/or inorganic, silicone and/or non-silicone materials resulting from the polymerization/crosslinking of a polymerizable/crosslinkable composition and/or from evaporation of the organic or aqueous solvent phase of a solution or dispersion of at least one polymeric material.

More precisely, it can be a multilayer film made by superposition of at least two, or more, layers obtained respectively by evaporation of the organic or aqueous solvent phase of solutions or dispersions of film-forming polymer(s) of different types.

### Adhesive

The adhesive employed in the method according to the invention can comprise a silicone adhesive selected from the silicone adhesives described above and/or a non-silicone adhesive.

In the case when the method employs a liquid composition containing the adhesive, this composition can contain, in addition to the adhesive, at least one organic or aqueous solvent selected from the solvents described above, in particular a volatile solvent which can be present in the liquid composition at a content in the range from 5 to 95 wt. % relative to the total weight of the composition, preferably from 10 to 85 wt. % and more preferably from 15 to 60 wt. %.

More particularly, these non-silicone adhesive materials can be selected from the adhesives of the “Pressure Sensitive Adhesives” type, for example those mentioned in the “Handbook of Pressure Sensitive Adhesive Technology” 3^{rd} edition, D. Satas.

These non-silicone adhesive materials are notably polymers selected from block or random copolymers comprising at least one monomer or an association of monomers for which the resultant polymer has a glass transition temperature below room temperature (25° C.), these monomers or associations of monomers being selected from butadiene, ethylene, propylene, isoprene, isobutylene and mixtures thereof. Examples of such materials are the block polymers such as styrene-butadiene-styrene, styrene-(ethylene-butylene)-styrene, styrene-isoprene-styrene such as those sold under the trade names “Kraton®” from SHELL CHEMICAL Co. Or “Vector®” from EXXON.

The non-silicone adhesive materials are in particular adhesive polymers selected from:

polyurethanes,
acrylic polymers,
butyl gums, notably polyisobutylenes,
ethylene-vinyl acetate polymers,
polyamides optionally modified with aliphatic chains,
natural gums,
and mixtures thereof.

They may in particular be adhesive copolymers resulting from the copolymerization of vinylic monomers with polymeric entities, for example those described in patent U.S. Pat. No. 6,136,296. The adhesive copolymers described in patent U.S. Pat. No. 5,929,173 possessing a polymer skeleton, with Tg varying from 0° C. to 45° C., grafted with chains derived from acrylic and/or methacrylic monomers and with, in contrast, a Tg varying from 50° C. to 200° C., may also be suitable for the invention.

The non-silicone adhesive materials are for example selected from the polyisobutylenes having a relative molar mass Mv greater than or equal to 10 000 and less than or equal to 150 000. In particular, this relative molar mass is greater than or equal to 18 000 and less than or equal to 150 000.

As commercial products that are particularly suitable for the present invention, we may mention the polyisobutylenes with relative molar mass Mv of 40 000, 55 000 and 85 000 respectively, sold under the respective trade names “Oppanol B 10®”, “Oppanol B 12®” and “Oppanol B 15®” by the company BASF, and mixtures thereof.

### Other Additives

i) Pigments and Colorants

The flexible sheet and/or the adhesive can in addition contain notably at least one colouring material, organic or inorganic, notably such as pigments or nacres conventionally used in cosmetic compositions.

By pigments, we mean white or coloured particles, mineral or organic, insoluble in the medium or aqueous, intended for colouring and/or opacifying the resultant film.

The pigments can be present at a rate of from 0.01 to 20 wt. %, notably from 0.01 to 15 wt. %, and in particular from 0.02 to 10 wt. %, relative to the total weight of the first composition and/or of the organic and/or inorganic film.

We may mention, as mineral pigments that can be used in the invention, the oxides of titanium, of zirconium or of cerium, as well as the oxides of zinc, of iron or of chromium, ferric blue, manganese violet, ultramarine and chromium hydroxide.

It can also be a pigment having a structure which can be for example of the sericite/brown iron oxide/titanium dioxide/silica type. Such a pigment is marketed for example under the reference COVERLEAF NS or JS by the company CHEMICALS AND CATALYSTS and has a contrast ratio close to 30.

The colouring matter can also comprise a pigment having a structure which can be, for example, silica microspheres containing iron oxide. An example of a pigment having this structure is that marketed by the company MIYOSHI under the reference PC BALL PC-LL-100 P, this pigment comprising silica microspheres containing yellow iron oxide.

Among the organic pigments that can be used in the invention, we may mention carbon black, pigments of the D & C type, lakes based on carmine, barium, strontium, calcium, aluminium or diketo-pyrrolopyrrole (DPP) described in documents EP-A-542669, EP-A-787730, EP-A-787731 and WO-A-96/08537.

“Nacres” are coloured particles of any shape, iridescent or not, notably produced in the shell of certain molluscs or alternatively synthesized, and which display a colour effect by optical interference.

The nacres can be selected from the nacreous pigments such as titanium mica coated with an iron oxide, mica coated with bismuth oxychloride, titanium mica coated with chromium oxide, titanium mica coated with an organic colorant as well as nacreous pigments based on bismuth oxychloride. They can also be mica particles, the surface of which is coated with at least two successive layers of metal oxides and/or organic colorants.

We may also mention, as example of nacres, natural mica coated with titanium dioxide, iron oxide, natural pigment or bismuth oxychloride.

Among the nacres that are available commercially, we may mention the nacres TIMICA, FLAMENCO and DUOCHROME (mica-based) marketed by the company ENGELHARD, the TIMIRON nacres marketed by the company MERCK, the PRESTIGE mica-based nacres marketed by the company ECKART and the SUNSHINE nacres based on synthetic mica, marketed by the company SUN CHEMICAL.

The nacres can more particularly possess a yellow, pink, red, bronze, orange, brown, golden and/or coppery colour or sheen.

As illustrative examples of nacres that can be used in the present invention, we may notably mention the gold-coloured nacres notably marketed by the company ENGELHARD under the name of Brillant gold 212G (Timica), Gold 222C (Cloisonne), Sparkle gold (Timica), Gold 4504 (Chromalite) and Monarch gold 233X (Cloisonne); the bronze nacres notably marketed by the company MERCK under the designation Bronze fine (17384) (Colorona) and Bronze (17353) (Colorona) and by the company ENGELHARD under the designation Super bronze (Cloisonne); the orange nacres notably marketed by the company ENGELHARD under the designation Orange 363C (Cloisonne) and Orange MCR 101 (Cosmica) and by the company MERCK under the designation Passion orange (Colorona) and Matte orange (17449) (Microna); the brown nacres notably marketed by the company ENGELHARD under the designation Nu-antique copper 340XB (Cloisonne) and Brown CL4509 (Chromalite); the nacres with a copper sheen notably marketed by the company ENGELHARD under the designation Copper 340A (Timica); the nacres with a red sheen notably marketed by the company MERCK under the designation Sienna fine (17386) (Colorona); the nacres with a yellow sheen notably marketed by the company ENGELHARD under the designation Yellow (4502) (Chromalite); the red nacres with a golden sheen notably marketed by the company ENGELHARD under the designation Sunstone G012 (Gemtone); the pink nacres notably marketed by the company ENGELHARD under the designation Tan opal G005 (Gemtone); the black nacres with a golden sheen notably marketed by the company ENGELHARD under the designation Nu antique bronze 240 AB (Timica), the blue nacres notably marketed by the company MERCK under the designation Matte blue (17433) (Microna), the white nacres with a silvery sheen notably marketed by the company MERCK under the designation Xirona Silver and the orange pink golden green nacres notably marketed by the company MERCK under the designation Indian summer (Xirona) and mixtures thereof.

The first composition and/or the organic and/or inorganic film according to the invention can also contain water-soluble or fat-soluble colorants at a content in the range from 0.01 to 10 wt. %, notably in the range from 0.01 to 5 wt. % relative to the total weight of the first composition or of the organic and/or inorganic film. The fat-soluble colorants are for example Sudan Red, DC Red 17, DC Green 6, β-carotene, soya oil, Sudan Brown, DC Yellow 11, DC Violet 2, DC Orange 5, quinoline yellow. Water-soluble colorants are for example beetroot juice, methylene blue.

ii) Special-Effect Material

The first composition and/or the organic and/or inorganic film according to the invention can contain at least one material with a specific optical effect, notably present in the organic and/or inorganic flexible film. This effect is different from a simple effect of conventional colouring, i.e. uniform and stabilized such as that produced by the conventional colorants described above, for example the monochromatic pigments. In the sense of the invention, “stabilized” signifies devoid of an effect of variability of colour with the angle of observation or in response to a temperature change.

This material is present in sufficient quantity to produce an optical effect that is perceptible with the naked eye. Advantageously, it is an effect selected from the goniochromatic, metallic and notably mirror, soft-focus, rainbow and/or thermochromic and/or photochromic effects. For example, this material can be selected from particles with a metallic sheen, goniochromatic colorants, diffracting pigments, thermochromic and photochromic agents, optical brighteners, as well as fibres, notably interference fibres. Of course, these various materials can be combined so as to produce two effects simultaneously, or even a novel effect according to the invention.

Particles with a Metallic Sheen

“Particles with a metallic sheen” denotes particles whose nature, size, structure and surface condition permit them to reflect incident light notably in a non-iridescent manner.

Particles having a substantially flat external surface are also suitable, as they can more easily give rise, if permitted by their size, their structure and their surface condition, to intense specular reflection, which can then be described as a mirror effect.

The particles with a metallic sheen that can be used in the invention, can for example reflect all the components of visible light without significantly absorbing one or more wavelengths. The spectral reflectance of these particles can for example be greater than 70% in the range 400-700 nm, and preferably at least 80%, or even 90% or 95%.

These particles generally have a thickness less than or equal to 1 μm, notably less than or equal to 0.7 μm, and in particular less than or equal to 0.5 μm.

The total proportion of particles with a metallic sheen is notably less than or equal to 20 wt. % and in particular less than or equal to 10 wt. % relative to the total weight of the first composition or of the organic and/or inorganic film.

The particles with a metallic sheen that can be used in the invention are in particular selected from:

particles of at least one metal and/or of at least one metallic derivative,
particles having a substrate, organic or mineral, monomaterial or multimaterial, coated at least partially with at least one layer with a metallic sheen, comprising at least one metal and/or at least one metallic derivative, and
mixtures of said particles.

Among the metals that can be present in said particles, we may mention for example Ag, Au, Cu, Al, Ni, Sn, Mg, Cr, Mo, Ti, Zr, Pt, Va, Rb, W, Zn, Ge, Te, Se and mixtures or alloys thereof. Ag, Au, Cu, Al, Zn, Ni, Mo, Cr, and mixtures or alloys thereof (for example bronzes and brasses) are the preferred metals.

“Metallic derivatives” denotes compounds derived from metals, notably oxides, fluorides, chlorides and sulphides.

Among the metallic derivatives that can be present in said particles, we may notably mention the metal oxides, for example the oxides of titanium, notably TiO₂, of iron, notably Fe₂O₃, of tin, of chromium, barium sulphate and the following compounds: MgF₂, CrF₃, ZnS, ZnSe, SiO₂, Al₂O₃, MgO, Y₂O₃, SeO₃, SiO, HfO₂, ZrO₂, CeO₂, Nb₂O₅, Ta₂O₅, MoS₂ and mixtures or alloys thereof.

According to a first variant, the particles with a metallic sheen can be composed of at least one metal as defined previously, of at least one metallic derivative as defined previously, or of one of the mixtures thereof.

These particles can be at least partially coated with a layer of another material, for example of transparent material such as notably rosin, silica, stearates, polysiloxanes, polyester resins, epoxy resins, polyurethane resins and acrylic resins.

As examples of these particles, we may mention aluminium particles, such as those marketed under the designations STARBRITE 1200 EAC® by the company SIBERLINE and METALURE® by the company ECKART.

We may also mention the metallic powders of copper or of alloy mixtures such as references 2844 marketed by the company RADIUM BRONZE, the metallic pigments such as aluminium or bronze, such as those marketed under the designations ROTOSAFE 700 by the company ECKART, the silica-coated aluminium particles marketed under the designation VISIONAIRE BRIGHT SILVER by the company ECKART and the metal alloy particles such as the silica-coated bronze powders (alloy of copper and zinc) marketed under the designation Visionaire Bright Natural Gold by the company Eckart.

According to a second variant, these particles can be particles having a substrate and which therefore have a multilayer, for example bilayer, structure. This substrate can be organic or mineral, natural or synthetic, monomaterial or multimaterial, filled or hollow. When the substrate is synthetic, it can be produced with a form that promotes the formation of a reflective surface after coating, notably after deposition of a layer of materials with a metallic sheen. The substrate can, for example, have a flat surface and the layer of materials with a metallic sheen can have an approximately uniform thickness.

In particular the substrate can be selected from the metals and the metallic derivatives as mentioned previously, and also from glasses, ceramics, aluminas, silicas, silicates and notably aluminosilicates and borosilicates, synthetic mica such as fluorophlogopite, and mixtures thereof, but this list is not limiting.

The layer with a metallic sheen can coat the substrate completely or partially, and this layer can be at least partially covered with a layer of another material, for example a transparent material notably such as mentioned previously. According to a particular embodiment, this layer with a metallic sheen coats the substrate completely, directly or indirectly, i.e. with interposition of at least one, metallic or nonmetallic, intermediate layer.

The metals or metallic derivatives that can be used in the reflective layer are as defined above. For example, it can be formed from at least one metal selected from silver, aluminium, chromium, nickel, molybdenum, gold, copper, tin, magnesium and mixtures thereof (alloys). Silver, chromium, nickel, molybdenum, and mixtures thereof, are used more particularly.

The following may be mentioned more particularly as examples of this second type of particles:

Glass particles coated with a metallic layer notably those described in documents JP-A-09188830, JP-A-10158450, JP-A-10158541, JP-A-07258460 and JP-A-05017710.

As examples of these particles with a glass substrate, we may mention those coated respectively with silver, gold or titanium, in the form of flakes, marketed by the company NIPPON SHEET GLASS under the designations MICROGLASS METASHINE. Particles with a glass substrate coated with silver, in the form of flakes, are sold under the designation MICROGLASS METASHINE REFSX 2025 PS by the company TOYAL. Particles with a glass substrate coated with nickel/chromium/molybdenum alloy are sold under the designation CRYSTAL STAR GF 550, GF 2525 by the same company. Those coated either with brown iron oxide, or titanium oxide, tin oxide or a mixture thereof are marketed under the designation REFLECKS® by the company ENGELHARD or under the reference METASHINE MC 2080GP by the company NIPPON SHEET GLASS.

These metal-coated glass particles can be coated with silica, such as those marketed under the designation METASHINE series PSS1 or GPS1 by the company NIPPON SHEET GLASS.

Spherical glass-substrate particles, metal-coated or uncoated, are notably sold under the designation PRIZMALITE MICROSPHERE by the company PRIZMALITE INDUSTRIES. Pigments from the METASHINE 1080R range marketed by the company NIPPON SHEET GLASS CO. LTD. are also suitable for the invention. These pigments, more particularly described in patent application JP 2001-11340, are flakes of C-GLASS containing 65 to 72% of SiO₂, coated with a layer of titanium dioxide of the rutile type (TiO₂). These glass flakes have an average thickness of 1 micron and an average size of 80 microns, giving a ratio of average size/average thickness of 80. They give blue, green, yellow or silvery reflections depending on the thickness of the layer of TiO₂.

Other particles have a borosilicate substrate coated with silver, and are also called “white nacres”.

Particles with a metallic substrate such as aluminium, copper, bronze, in the form of flakes, are sold under the trade name STARBRITE by the company SILBERLINE and under the name VISIONAIRE by the company ECKART.

Particles having a substrate of synthetic mica coated with titanium dioxide, and for example with particle size between 80 and 100 μm, with a substrate of synthetic mica (fluorophlogopite) coated with titanium dioxide representing 12% of the total weight of the particle, are sold under the designation PROMINENCE by the company NIHON KOKEN. Particles with a metallic sheen can also be selected from particles formed from a stack of at least two layers with different refractive indices. These layers can be of a polymeric or metallic nature and can notably include at least one polymeric layer.

Thus, particles with a metallic effect can be particles derived from a multilayer polymer film. The materials for constituting the various layers of the multilayer structure are of course selected in such a way as to endow the particles thus formed with the desired metallic effect. Such particles are notably described in WO 99/36477, U.S. Pat. No. 6,299,979 and U.S. Pat. No. 6,387,498 and are more particularly identified below in the goniochromatic section.

Diffracting Pigments

By “diffracting pigment” we mean, in the sense of the present invention, a pigment capable of producing a colour variation according to the angle of observation when lit by white light, owing to the presence of a light-diffracting structure.

A diffracting pigment can comprise a diffraction grating, capable for example of diffracting an incident ray of monochromatic light in defined directions.

The diffraction grating can comprise a regularly repeating unit, notably a line, the distance between two adjacent units being of the same order of magnitude as the wavelength of the incident light.

When the incident light is polychromatic, the diffraction grating will separate the different spectral components of the light and produce a rainbow effect.

Regarding the structure of the diffracting pigments, it may be useful to refer to the article “Pigments Exhibiting Diffractive Effects” of Alberto Argoitia and Matt Witzman, 2002, Society of Vacuum Coaters, 45^{th} Annual Technical Conference Proceedings 2002.

The diffracting pigment can be produced with units having different profiles, notably triangular, symmetrical or asymmetric, with gaps, of constant or variable width, sinusoidal. The spatial frequency of the grating and the depth of the units will be selected in relation to the degree of separation of the various orders desired. The frequency can vary for example between 500 and 3000 lines per mm.

Preferably, the particles of the diffracting pigment each have a flattened shape, and notably are in the form of flakes.

One and the same pigment particle can have two crossed diffraction gratings, perpendicular or otherwise.

A possible structure for the diffracting pigment can comprise a layer of a reflective material, covered at least on one side with a layer of a dielectric material. The latter can endow the diffracting pigment with improved rigidity and durability. The dielectric material can be selected for example from the following materials: MgF₂, SiO₂, Al₂O₃, AlF₃, CeF₃, LaF₃, NdF₃, SmF₂, BaF₂, CaF₂, LiF and their combinations. The reflective material can be selected for example from the metals and their alloys and also from the nonmetallic reflective materials. Among the metals that can be used, we may mention Al, Ag, Cu, Au, Pt, Sn, Ti, Pd, Ni, Co, Rd, Nb, Cr and their compounds, combinations or alloys. Such a reflective material can, by itself, constitute the diffracting pigment, which will then be monolayered.

As a variant, the diffracting pigment can comprise a multilayer structure having a core of a dielectric material coated with a reflective layer on at least one side, or even completely encapsulating the core. A layer of a dielectric material can also cover the reflective layer or layers. The dielectric material used is then preferably inorganic, and can be selected for example from the metal fluorides, metal oxides, metal sulphides, metal nitrides, metal carbides and their combinations. The dielectric material can be in the crystalline, semi-crystalline or amorphous state. The dielectric material, in this configuration, can for example be selected from the following materials: MgF₂, SiO, SiO₂, Al₂O₃, TiO₂, WO, AlN, BN, B₄C, WC, TiC, TiN, N₄Si₃, ZnS, glass particles, carbon particles of the diamond type and their combinations.

The diffracting pigment used can notably be selected from those described in US patent application US 2003/0031870 published on 13 Feb. 2003.

A diffracting pigment can comprise for example the following structure: MgF₂/Al/MgF₂, a diffracting pigment having this structure being marketed under the designation SPECTRAFLAIR 1400 Pigment Silver by the company FLEX PRODUCTS, or SPECTRAFLAIR 1400 Pigment Silver FG. The proportion by weight of MgF₂ can be between 80 and 95% of the total weight of the pigment.

Goniochromatic Colouring Agents

In the sense of the invention, a goniochromatic colouring agent can exhibit a colour change, also called “colour flop”, depending on the angle of observation, greater than that encountered with nacres. One or more goniochromatic colouring agents can be used simultaneously.

The goniochromatic colouring agent can be selected such that it exhibits a relatively large colour change with the angle of observation.

The goniochromatic colouring agent can thus be selected so as to be able to observe, for a variation of the angle of observation between 0° and 80° under illumination at 45°, a colour change ΔE of the cosmetic composition, measured in the colorimetric space CIE 1976, of at least 2.

The goniochromatic colouring agent can also be selected so as to be able to observe, for illumination at 45° and variation of the angle of observation between 0° and 80°, a change Dh in the angle of tint of the cosmetic composition, in the CIE 1976 plane, of at least 30° and even at least 40° or at least 60°, and even of at least 100°.

The goniochromatic colouring agent can be selected for example from multilayer interference structures and liquid crystal colorants.

In the case of a multilayer structure, the latter can comprise for example at least two layers, each layer, independently or not of the other layer or layers, being made for example from at least one material selected from the group comprising the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MoS₂, cryolite, alloys, polymers and their combinations.

The multilayer structure may or may not exhibit, relative to a central layer, symmetry with respect to the chemical nature of the stacked layers.

Examples of symmetrical multilayer interference structures that can be used in compositions made in accordance with the invention are for example the following structures: Al/SiO₂/Al/SiO₂/Al, pigments having this structure being marketed by the company DUPONT DE NEMOURS; Cr/MgF₂/Al/MgF₂/Cr, pigments having this structure being marketed under the designation CHROMAFLAIR by the company FLEX; MoS₂/SiO₂/Al/SiO₂/MoS₂; Fe₂O₃/SiO₂/Al/SiO₂/Fe₂O₃, and Fe₂O₃/SiO₂/Fe₂O₃/SiO₂/Fe₂O₃, pigments having these structures being marketed under the designation SICOPEARL by the company BASF; MoS₂/SiO₂/mica-oxide/SiO₂/MoS₂; Fe₂O₃/SiO₂/mica-oxide/SiO₂/Fe₂O₃; TiO₂/SiO₂/TiO₂ and TiO₂/Al₂O₃/TiO₂; SnO/TiO₂/SiO₂/TiO₂/SnO; Fe₂O₃/SiO₂/Fe₂O₃; SnO/mica/TiO₂/SiO₂/TiO₂/mica/SnO, pigments having these structures being marketed under the designation XIRONA by the company MERCK (Darmstadt). As examples, these pigments can be the pigments of silica/titanium dioxide/tin oxide structure marketed under the name XIRONA MAGIC by the company MERCK, the pigments of silica/brown iron oxide structure marketed under the name XIRONA INDIAN SUMMER by the company MERCK and the pigments of silica/titanium dioxide/mica/tin oxide structure marketed under the name XIRONA CARIBBEAN BLUE by the company MERCK. We may also mention the pigments INFINITE COLORS from the company SHISEIDO. Different effects are obtained, depending on the thickness and the nature of the different layers. Thus, with the structure Fe₂O₃/SiO₂/Al/SiO₂/Fe₂O₃ there is transition from golden green to grey-red for SiO₂ layers from 320 to 350 nm; from red to golden for SiO₂ layers from 380 to 400 nm; from violet to green for SiO₂ layers from 410 to 420 nm; from copper to red for SiO₂ layers from 430 to 440 nm.

It is also possible to use goniochromatic colouring agents of multilayer structure comprising alternating polymeric layers.

To illustrate the materials that can constitute the various layers of the multilayer structure, we may mention, as a non-limiting list: polyethylene naphthalate (PEN) and its isomers for example 2,6-, 1,4-, 1,5-, 2,7- and 2,3-PEN, polyalkylene terephthalates, polyimides, polyetherimides, atactic polystyrenes, polycarbonates, alkyl polymethacrylates and polyacrylates, syndiotactic polystyrene (sPS), syndiotactic poly-alpha-methylstyrene, syndiotactic polydichlorostyrene, copolymers and mixture of its polystyrenes, cellulose derivatives, polyalkylene polymers, fluorinated polymers, chlorinated polymers, polysulphones, polyethersulphones, polyacrylonitriles, polyamides, silicone resins, epoxy resins, polyvinyl acetate, polyether-amides, ionomeric resins, elastomers and polyurethanes. Copolymers are also suitable, for example copolymers of PEN (for example, copolymers of 2,6-, 1,4-, 1,5-, 2,7-, and/or 2,3-naphthalene dicarboxylic acid or its esters with (a) terephthalic acid or its esters; (b) isophthalic acid or its esters; (c) phthalic acid or its esters; (d) alkane glycols; (e) cycloalkane glycols (for example cyclohexane dimethanol diol); (f) alkane dicarboxylic acids; and/or (g) cycloalkane dicarboxylic acids, copolymers of polyalkylene terephthalates and styrene copolymers. In addition, each individual layer can include mixtures of two or more of the preceding polymers or copolymers. The materials for constituting the various layers of the multilayer structure are of course selected in such a way as to endow the particles thus formed with the desired optical effect.

We may mention, as examples of pigments with a polymeric multilayer structure, those marketed by the company 3M under the designation COLOR GLITTER.

The liquid crystal colorants comprise for example silicones or cellulose ethers, onto which mesomorphic groups are grafted.

The liquid crystal goniochromatic particles used can be, for example, those sold by the company CHENIX as well as those marketed under the designation HELICONE® HC by the company WACKER.

These agents can also be in the form of dispersed goniochromatic fibres. Such fibres can for example have a size between 50 μm and 700 μm, for example of about 300 μm.

In particular, interference fibres with multilayer structure can be used. Polymer fibres with multilayer structure are notably described in documents EP-A-921217, EP-A-686858 and U.S. Pat. No. 5,472,798. The multilayer structure can comprise at least two layers, each layer, independently or not of the other layer or layers, being made from at least one synthetic polymer. The polymers present in the fibres can have a refractive index in the range from 1.30 to 1.82 and preferably in the range from 1.35 to 1.75. The preferred polymers for constituting the fibres are polyesters such as polyethylene terephthalate, polyethylene naphthalate, polycarbonate; acrylic polymers such as polymethyl methacrylate; polyamides. Goniochromatic fibres with bilayer structure, polyethylene terephthalate/nylon-6, are marketed by the company TEIJIN under the designation MORPHOTEX.

As a variant, this goniochromatic colouring agent can be combined with at least one diffracting pigment.

The combination of these two materials results in a composition or a film which exhibits increased variability of colour, and which therefore allows an observer to perceive a colour change, or even a movement of colour, in numerous conditions of observation and of illumination.

The weight ratio of the diffracting pigment relative to the goniochromatic colouring agent is preferably between 85/15 and 15/85, more preferably between 80/20 and 20/80, and even more preferably between 60/40 and 40/60, for example of the order of 50/50. Such a ratio favours the production of a sustained rainbow effect and goniochromatic effect.

Optical Brighteners

The optical brighteners are compounds that are familiar to a person skilled in the art. Such compounds are notably described in “Fluorescent Whitening Agent, Encyclopedia of Chemical Technology, Kirk-Othmer”, vol 11, p. 227-241, 4^{th} edition, 1994, Wiley.

They can be defined more particularly as compounds which essentially absorb in the UVA between 300 and 390 nm and reemit essentially between 400 and 525 nm.

Among the optical brighteners, we may mention more particularly the derivatives of stilbene, in particular the polystyrylstilbenes and the triazinestilbenes, the coumarin derivatives, in particular the hydroxycoumarins and the aminocoumarins, the oxazole, benzoxazole, imidazole, triazole, and pyrazoline derivatives, the pyrene derivatives and the porphyrin derivatives and mixtures thereof.

Such compounds are readily available commercially. We may mention for example:

the stilbene derivative of naphtho-triazole sold under the trade name “Tinopal GS”, disodium di-styryl-4,4′ biphenyl sulphonate (CTFA name: disodium distyrylbiphenyl disulphonate) sold under the trade name “Tinopal CBS-X”, the cationic derivative of aminocoumarin sold under the trade name “Tinopal SWN CONC.”, 4,4′-bis[(4,6-dianilino-1.3,5-triazin-2-yl)amino]stilbene-2,2′-sodium disulphonate sold under the trade name “Tinopal SOP”, 4,4′-bis-[(4-anilino-6-bis(2-dydroxyethyl)amino-1,3,5-triazin-2-yl)amino]stilbene-2,2′-disulphonic acid sold under the trade name “Tinopal UNPA-GX”, 4,4′-bis-[anilino-6-morpholine-1,3,5-triazin-2-yl)amino]stilbene sold under the trade name “Tinopal AMS-GX”, 4,4′-bis-[(4-anilino-6-(2-hydroxyethyl)methyl amino-1,3,5-triazin-2-yl)amino]stilbene-2,2′-disodium sulphonate sold under the trade name “Tinopal 5BM-GX”, all from the company CIBA Spécialités Chimiques,
2,5 thiophene di-yl bis(5 ter-butyl-1,3 benzoxazole) sold under the trade name “Uvitex OB” by the company CIBA,
the anionic derivative of di-aminostilbene in dispersion in water sold under the trade name “Leucophor BSB liquid” by the company CLARIANT,
the optical brightener lakes sold under the trade name “COVAZUR” by the company WACKHERR.

The optical brighteners that can be used in the present invention can also be in the form of copolymers, for example of acrylates and/or of methacrylates, grafted with optical brightener groups as described in application FR 99 10942.

They can be used as they are or can be incorporated in the film in the form of particles and/or fibres coated with said optical brightener, such as those described below.

In particular, fibres coated with optical brightener can be used, such as those marketed by the company LCW under the trade reference Fiberlon 54 ZO3, having a length of about 0.4 mm and a thickness of 0.5 denier.

Material with Relief Effect:

The relief effect may or may not be associated with an optical effect. A material of this type is generally present in a sufficient amount to confer a relief effect that is perceptible to the touch or even to the naked eye. It can notably be a rough and/or hammered effect.

### Material Imparting a Rough Appearance

Thus, the flexible sheet according to the invention is particularly advantageous for the fixing of solid particles or fibres in its film, thus providing make-up with original relief. Furthermore, particles of approximately spherical or oval shape can give the make-up a soft feel.

Advantageously, the solid particles have an approximately spherical shape, to allow them to be distributed well during application on the first layer.

The solid particles used according to the invention can have an average size in the range from 2.5 μm to 5 mm, and preferably from 50 μm to 2 mm. The smaller the particles, the more satisfactory the lasting properties of the particles. The use of particles is also compatible with the production of patterns.

The solid particles can be of any material satisfying the properties of density defined previously. For example, the solid particles can be of a material selected from glass, zirconium oxide, tungsten carbide, plastics such as polyurethanes, polyamides, polytetrafluoroethylene, polypropylene, metals such as steel, copper, brass, chromium; marble, onyx, jade, natural mother-of-pearl, precious stones (diamond, emerald, ruby, sapphire), amethyst, aquamarine. Glass beads are preferably used, such as those sold under the designation “SILIBEADS®” by the company SIGMUND LINDNER; these beads have the additional advantage of also imparting a glossy and sparkling effect to the make-up. The solid particles, deformable or not, can be full or hollow, colourless or coloured, coated or uncoated.

The fibres that can be used according to the invention can be fibres of synthetic or natural origin, mineral or organic.

By “fibre” we mean an object of length L and diameter D such that L is much greater than D, D being the diameter of the circle in which the fibre cross-section can be inscribed. In particular, the ratio L/D (or form factor) is selected in the range from 3.5 to 2500, preferably from 5 to 500, and more preferably from 5 to 150.

Notably they may be fibres used in the manufacture of textiles and notably fibres of silk, cotton, wool, flax, cellulose fibres, notably extracted from wood, vegetables or algae, rayon, polyamide (Nylon®), viscose, acetate notably rayon acetate, poly-(p-phenylene-terephthalamide) (or aramid) notably Kevlar®, acrylic polymer notably polymethyl methacrylate or poly(2-hydroxyethyl methacrylate), polyolefin and notably polyethylene or polypropylene, glass, silica, carbon notably in the form of graphite, polytetrafluoroethylene (such as Teflon®), insoluble collagen, polyesters, polyvinyl chloride or vinylidene, polyvinyl alcohol, polyacrylonitrile, chitosan, polyurethane, polyethylene phthalate, fibres formed from polymer blends such as those mentioned previously, such as polyamide/polyester fibres.

### Material Imparting a Hammered Appearance

The inventors also found that it was possible to incorporate, in the flexible sheet employed in the invention, a material comprising a mixture of pyrogenic silica, metallic pigment and organopolysiloxane compound to endow it with a hammered appearance.

Such a mixture is notably described in patent application EP 1 040 813.

Material with Olfactory Effect

Advantageously, the organic and/or inorganic film according to the invention can also be endowed with olfactory properties notably by incorporating, in said film, at least one sweet-smelling material or a perfume.

The perfume can be selected from any odoriferous substance well known by a person skilled in the art, and notably from the essential oils and/or the essences.

This olfactory material can, if necessary, be incorporated via a solvent-plasticizer.

By “solvent-plasticizer” we mean a compound which dissolves the olfactory material at least partially and which is able to evaporate slowly.

The solvent-plasticizer can be selected from glycols such as dipropylene glycol, ethyldiglycol, n-propylglycol, n-butylglycol, methyldiglycol, n-butyldiglycol; alcohols such as cyclohexanol, ethyl-2 butanol, methoxy-3 butanol, ethyl-2 hexanol, phenoxyethanol; esters, such as glycol monoacetate, ethylglycol acetate, n-butylglycol acetate, ethyldiglycol acetate, n-butyldiglycol acetate, methyl abietate, isopropyl myristate, propylene glycol diacetate, methyl ether acetate of propylene glycol; glycol ethers such as methyl ether of dipropylene glycol, butyl ether of dipropylene glycol, alone or mixed.

The flexible sheet and/or the adhesive can also contain one or more formulation additives commonly used in cosmetics and more especially in the nail cosmetic and/or nail care area. They can notably be selected from vitamins, trace elements, emollients, sequestering agents, alkalizing or acidifying agents, wetting agents, thickeners, dispersants, anti-foaming agents, spreading agents, co-resins, preservatives, UV filters, actives, moisturizers, neutralizing agents, stabilizers, antioxidants and mixtures thereof.

Thus, they can notably incorporate, as actives, hardening or strengthening agents for keratinous materials, actives promoting nail growth such as methylsulphonylmethane and/or actives for treating various disorders localized at the nails, for example antimycotics or antimicrobials.

The amounts of these various ingredients are those conventionally used in this field and are for example from 0.01 to 20 wt. %, and notably from 0.01 to 10 wt. % relative to the total weight of the flexible sheet and/or of the adhesive.

The invention is illustrated in more detail with the following example. Unless stated otherwise, the amounts stated are expressed in percentage by weight.

### EXAMPLE

A flexible article according to a particular embodiment of the invention could be produced as follows:

Flexible article comprising a layer of polymeric material resulting from the evaporation of a composition comprising 20% of DC217 Flake Resin phenylsilicone film-forming resin, ethyl acetate and butyl acetate solvent mixture (50/50) and 1% of pigments. The film thus obtained is coated with an adhesive: BIOPSA 7-4600 (from the company Dow Corning) used at 100%. A fluorinated counter-adhesive is then applied on the adhesive material.

1. A method for make-up and/or care of the nails comprising gluing onto the nail, by means of an adhesive, a flexible sheet comprising at least one layer of at least one organic and/or inorganic material, the organic and/or inorganic material and/or the adhesive containing at least one silicone compound.

2. A method for make-up and/or care of the nails comprising gluing onto the nail, by means of an adhesive, a flexible sheet comprising at least one layer of at least one organic and/or inorganic material, the organic and/or inorganic material and the adhesive each containing at least one silicone compound, and wherein said sheet has a thickness in the range from 1 μm to 2 mm.

3. The method according to claim 1 or 2, wherein the sheet, once applied on the nail, can be removed with the aid of a solvent that is able to dissolve, at least partially, said organic and/or inorganic material and the adhesive.

4. The method according to claim 1, wherein the flexible sheet has a first face intended to be in contact with the nail, and a second face opposite the first, the adhesive being disposed on the first face of the sheet in such a way that the first face is self-adhesive.

5. The method according to claim 1, further comprising:
a. applying at least one layer of a liquid or solid composition containing said adhesive on the nail; and
b. applying, on the layer containing the adhesive, one face of said sheet so as to glue the latter on the nail.

6. The method according to claim 1, further comprising:
a. coating a first face of the flexible sheet with a liquid or solid composition containing said adhesive; and
b. bringing the first face thus coated into contact with the nail so as to glue said sheet on the nail.

7. The method according to claim 1 wherein the silicone compound represents from 5 to 100 wt. % relative to the total weight of the layer containing it or of the adhesive.

8. The method according to claim 1, wherein the organic and/or inorganic material of the sheet comprises at least one film-forming silicone compound.

9. The according to claim 1, wherein the adhesive comprises at least one adhesive silicone material.

10. The method according to claim 1, wherein the organic and/or inorganic material and the adhesive each contain at least one silicone compound.

11. The method according to claim 1, wherein the silicone compound is selected from the group consisting of film-forming silicone compounds, silicone materials displaying adhesive properties and mixtures thereof.

12. The method according to claim 11, wherein the film-forming silicone compound is selected from the group consisting of silicone resins, polymers obtained by supramolecular assembly, crosslinked polymers of silicone, organic/silicone hybrid polymers obtained by sol gel technology, organic/silicone hybrid polymers, dendrimers of silicones and mixtures thereof.

13. The method according to claim 12, wherein the film-forming silicone compound is a silicone resin.

14. The method according to claim 13, wherein the silicone resin is at least one phenylsilicone resin with free silanol groups and mixtures thereof.

15. The method according to claim 9, wherein the at least one silicone adhesive material is selected from the group consisting of silicone resins, silicone elastomers, organic/silicone hybrid copolymers and mixtures thereof.

16. The method according to claim 15, wherein the at least one silicone adhesive material is selected from pressure-sensitive silicone resins obtained by reaction of a silicone resin with reactive SiOH end groups and of a fluid polyorganosiloxane polymer with viscosity in the range from 1000 to 200000 cSt bearing reactive SiOH end groups.

17. The method according to claim 1 wherein the silicone compound is selected from the group consisting of
phenylsilicone resins with free silanol groups,
“room temperature vulcanization” reactive elastomeric silicones with adhesive properties, selected from the group consisting of
silicone fluids or gums bearing alkoxysilane, acetoxysilane or silanol groups for in situ condensation/crosslinking in the presence of water and optionally a catalyst,
the silicone fluids or gums bearing SIH groups, designated “A”, mixed with silicones with unsaturated CH═CH₂ groups, designated “B”, the groups A crosslinking with B on the substrate by hydrosilylation with a platinum or tin catalyst,
and mixtures thereof.

18. A method for make-up and/or care of the nails comprising gluing onto the nail, by means of an adhesive, a flexible sheet comprising at least one layer of at least one organic and/or inorganic material, the organic and/or inorganic material and/or the adhesive containing at least one silicone compound selected from the group consisting of:
pressure-sensitive silicone resins obtained by reaction of a silicone resin with reactive SiOH end groups and of a fluid polyorganosiloxane polymer with viscosity in the range from 1000 to 200000 cSt bearing reactive SIOH end groups,
phenylsilicone resins with free silanol groups,
“room temperature vulcanization” reactive elastomeric silicones with adhesive and/or film-forming properties selected from the group consisting of:
silicone fluids or gums bearing alkoxysilane, acetoxysilane (or silanol) groups for in situ condensation/crosslinking in the presence of water and optionally a catalyst,
silicone fluids or gums bearing SiH groups, designated “A”, mixed with silicones with unsaturated CH═CH₂ groups, designated “B”, the groups A crosslinking with B on the substrate by hydrosilylation with a platinum or tin catalyst,
and mixtures thereof.

19. The method according to claim 18 wherein the flexible sheet comprising at least one organic and/or inorganic material is a polymerized/crosslinked film resulting from the polymerization/crosslinking of a crosslinkable composition and/or a film resulting from the evaporation of the organic or aqueous solvent phase of a solution or dispersion of at least said organic and/or inorganic material.

20. The method according to claim 19, wherein the flexible sheet possesses a dry extract greater than 80% wt. %.

21. The method according to claim 1, wherein the flexible sheet results from at least one selected from the group consisting of thermal, photochemical and chemical polymerization/crosslinking of a polymerizable/crosslinkable composition.

22. The method according to claim 1 wherein the flexible sheet results from the evaporation of the solvent phase of a solution or dispersion of at least one organic and/or inorganic film-forming material.

23. The method according to claim 22, wherein the organic and/or inorganic material is selected from the group consisting of film-forming silicone compounds and non-silicone film-forming polymers.

24. The method according to claim 23, wherein the non-silicone film-forming polymer is selected from the group consisting of homo- and co-polymer esters and/or amides of (meth)acrylic acids, homo- and co-polymer vinyl esters or amides, celluloses and cellulose derivatives, polyurethanes, acrylic polyurethanes, polyureas, polyurea polyurethanes, polyester polyurethanes, polyether polyurethanes, polyesters, polyester-amides, aliphatic-chain polyesters, epoxides, and arylsulphonamide condensates.

25. The method according to claim 1, wherein the flexible sheet results from the evaporation of the aqueous phase of an aqueous dispersion of particles of film-forming polymer(s).

26. The method according to claim 25, wherein the aqueous dispersion of particles of film-forming polymer(s) is a latex, a pseudolatex or a mixture thereof.

27. Method The method according to claim 26, wherein said film-forming polymer is selected from the group consisting of polycondensates, anionic, cationic, non-ionic or amphoteric polyurethanes, polyurethane-acrylics, polyurethane-polyvinylpyrrolidones, polyester-polyurethanes, polyether-polyurethanes, polyureas, polyurea polyurethanes, polyesters, polyester amides, aliphatic-chain polyesters, polyamides and epoxyester resins, acrylic and/or vinylic polymers or copolymers, nitrocellulose/acrylic copolymers, polymers resulting from the radical polymerization of one or more radical monomers within and/or partially on the surface, of pre-existing particles of at least one polymer selected from the group comprising polyurethanes, polyureas, polyesters and polyesteramides and alkyds.

28. The method according to claim 1, wherein the adhesive material further comprises a non-silicone adhesive.

29. The method according to claim 28, wherein the non-silicone adhesive is selected from the group consisting of copolymers resulting from the copolymerization of vinylic monomers with polymeric entities, copolymers possessing a polymeric skeleton, with Tg varying from 0° C. to 45° C., grafted with chains derived from acrylic and/or methacrylic monomers and possessing a Tg varying from 50° C. to 200° C. and the polyisobutylenes with a relative molar mass My greater than or equal to 10 000 and less than or equal to 150 000.

30. The method according to claim 1, wherein the flexible sheet and/or the adhesive further comprises at least one formulation additive selected from the group consisting of co-resins, plasticizers, coalescence agents and spreading agents.

31. The method according to claim 1, wherein the flexible sheet and/or the flexible adhesive material further comprise at least one colorant.

32. The method according to claim 1, wherein the flexible sheet and/or the adhesive further comprise at least one material with an effect selected from the group consisting of an optical effect, a relief effect and an olfactory effect.

33. The method according to claim 1, wherein the adhesive is transparent.

34. The method according to claim 1, further comprising, after application of the article on the nail, coating a face of the article opposite that in contact with the nail with at least one layer of a liquid composition comprising a film-forming polymer and an organic solvent.

35. Article for make-up and/or care of the nails comprising a flexible sheet with at least one layer of at least one organic and/or inorganic material, said sheet having a first adhesive face with an adhesive and intended to be brought into contact with the nail, and a second face opposite the first, the organic and/or inorganic material and/or the adhesive containing at least one silicone compound.

36. An article for make-up and/or care of the nails comprising a flexible sheet with at least one layer of at least one organic and/or inorganic material, said sheet having a first adhesive face with an adhesive and intended to be brought into contact with the nail, and a second face opposite the first, the organic and/or inorganic material and/or the adhesive each containing at least one silicone compound wherein a thickness of said flexible sheet is in a range from 1 μm to 2 mm.

37. An article for make-up and/or care of the nails comprising a flexible sheet with at least one layer of at least one organic and/or inorganic material, said sheet having a first adhesive face with an adhesive and intended to be brought into contact with the nail, and a second face opposite the first, the organic and/or inorganic material and/or the adhesive comprises at least one silicone compound selected from the group consisting of
pressure-sensitive silicone resins obtained by reaction of a silicone resin with reactive SiOH end groups and of a fluid polyorganosiloxane polymer with viscosity in the range from 1000 to 200000 cSt bearing reactive SiOH end groups,
phenylsilicone resins with free silanol groups,
“room temperature vulcanization” reactive elastomeric silicones with adhesive and/or film-forming properties selected from the group consisting of
silicone fluids or gums bearing alkoxysilane, acetoxysilane, and silanol groups for in situ condensation/crosslinking in the presence of water and optionally a catalyst,
silicone fluids or gums bearing SiH groups, designated “A”, mixed with silicones with unsaturated CH═CH₂ groups, designated “B”, the groups A crosslinking with B on the substrate by hydrosilylation with a platinum or tin catalyst,
and mixtures thereof.

38. The article according to claim 35 further comprising a protective film in contact with the first face of the flexible sheet, wherein the protective film is to be removed prior to placing the article on the nail.

39. The article according to claim 38, wherein a face of the protective film in contact with the first face of the sheet is covered with a non-stick material.

40. The article according to claim 35, wherein the organic and/or inorganic material and the adhesive each comprise at least one silicone compound.

41. The article according to claim 40, wherein the organic and/or inorganic material is a silicone resin selected from the group consisting of phenylsilicone resins with free SiOH groups and mixtures thereof.

42. The article according to claim 38, wherein the adhesive is selected from the group consisting of silicone resins and elastomers, organic/silicone hybrid copolymers and mixtures thereof.

43. The article according to claim 42, wherein the adhesive is selected from the group consisting of pressure-sensitive silicone resins obtained by reaction of a silicone resin with reactive SiOH end groups and a fluid polyorganosiloxane polymer with viscosity in the range from 1000 to 200000 cSt bearing reactive SiOH end groups.

44. Kit for make-up and/or care of the nails comprising:
a) a flexible sheet with at least one layer of at least one organic or inorganic material, said flexible sheet having a first face intended to be brought into contact with the nail, and a second face opposite the first, said sheet having a thickness in the range from 1 μm to 2 mm, and preferably, in the range from 1 μm to 1 mm.
b) a liquid or solid composition containing at least one adhesive,
the adhesive and/or the organic and/or inorganic material containing at least one silicone compound.

45. The make-up kit according to claim 44, wherein the organic and/or inorganic material and the adhesive contain at least one silicone compound.

46. The make-up kit according to claim 45, wherein the organic and/or inorganic material is a silicone resin selected from the group consisting of phenylsilicone resins with free SiOH groups and mixtures thereof.

47. The make-up kit according to claim 45, wherein the adhesive is selected from the group consisting of silicone resins and elastomers, organic/silicone hybrid copolymers and mixtures thereof.

48. The make-up kit according to claim 47, wherein the adhesive is selected from the group consisting of pressure-sensitive silicone resins obtained by reaction of a silicone resin with reactive SiOH end groups and of a fluid polyorganosiloxane polymer with viscosity in the range from 1000 to 200000 cSt bearing reactive SiOH end groups.
